(19) United States
(12) Patent Application Publication
(10) Pub. No.: US 2016/0257918 A1
(43) Pub. Date: Sep. 8, 2016

(54) Generation and Selection of Embryos in Vitro

(71) Applicant: Berkeley Lights, Inc., Emeryville, CA (US)

(72) Inventors: Kevin Chapman, Santa Monica, CA (US); Volker Kurz, Castro Valley, CA (US); Peggy Radel, Berkeley, CA (US); Grant Yonehiro, Woodside, CA (US)

(21) Appl. No.: 15/061,295

(22) Filed: Mar. 4, 2016

Related U.S. Application Data
(60) Provisional application No. 62/128,458, filed on Mar. 4, 2015.

(51) Int. Cl. C12M 3/00; C12M 3/06; C12M 1/34; C12M 1/00

(52) U.S. Cl. CPC C12M 21/06; C12M 45/07; C12M 23/16; C12M 41/46

(57) ABSTRACT

Methods of improved monitoring, testing and/or culturing of ova and/or sperm for in vitro fertilization are described herein. Methods of improved monitoring, testing and/or culturing of embryos during ex vivo pre-implantation selection are also described. The ova, sperm or embryos may be derived from wild animals or zoo animals. The ova, sperm or embryos can be mammalian, such as human, bovine, porcine, ovine, caprine, equine, canine, feline, murine, or the like.

This application is a non-provisional application claiming the benefit under 35 U.S.C. 119(e) of U.S. Provisional Application No. 62/128,458 filed on Mar. 4, 2015, which disclosure is herein incorporated by reference in its entirety.

### BACKGROUND OF THE INVENTION

Infertility is a serious problem for a large number of people. In vitro fertilization is often unsuccessful both in terms of failure to a viable pregnancy as well as producing an unacceptably high rate of multiple births with the incumbent risks. Methods of selecting embryos pre-implantation leading to a high probability of successful single implantation would be highly advantageous.

### SUMMARY OF THE INVENTION

Some embodiments of the invention relate to improved monitoring, testing and/or culturing of embryos during ex vivo pre-implantation selection. In such embodiments, the morphology, internal markers, surface markers, and/or secretions from a single embryo or a group of embryos can be monitored. The embryos can be animal embryos, such as livestock embryos or embryos derived from wild animals or zoo animals. The embryos can be mammalian embryos, such as human, bovine, porcine, ovine, caprine, equine, canine, feline, murine embryos, or the like.

Some embodiments of the invention relate to improved monitoring, testing and/or culturing of ova and/or sperm for in vitro fertilization. In some embodiments, the morphology, internal markers, surface markers, and/or secretions from a single ova or a group of ova can be monitored. In some embodiments, the morphology, internal markers, surface markers, secretions, and/or motility of a single sperm or a group of sperm can be monitored. The ova and sperm can be obtained from one or more animals, such as livestock, wild animals, and/or zoo animals. The ova and sperm can be obtained from a mammal, such as a human, cow, pig, sheep, goat, horse, dog, cat, mouse, or the like.

Some embodiments of the invention are directed to devices that allow selecting, from a group of embryos, an embryo that is able to produce a viable pregnancy. Some related embodiments are directed to devices that facilitate fertilization of ova and/or selecting, from a group of ova, an egg that is able to produce a viable embryo. Other related embodiments are directed to devices that facilitate fertilization of ova and/or selecting, from a group of sperm, a sperm that is able to produce a viable embryo.

In one aspect, a process for producing an embryo in a microfluidic device is provided, where the process includes introducing an ovum into an isolation pen of the microfluidic device; introducing at least one sperm into the microfluidic device; allowing the at least one sperm to contact the ovum under conditions conducive to fertilization of the ovum; and incubating the contacted ovum and the at least one sperm in the microfluidic device for a period of time at least long enough for the ovum and the at least one sperm to form the embryo.

In various embodiments, introducing the ovum into the isolation pen may include using a dielectrophoresis (DEP) force. The DEP force may be produced by an optoelectronic tweezers (OET) configuration. In various embodiments, introducing the at least one sperm into the isolation pen may include using a dielectrophoresis (DEP) force. The DEP force may be produced by an optoelectronic tweezers (OET) configuration.

In various embodiments, introducing the ovum into the isolation pen may include using electro-wetting force. The electro-wetting force may be produced by an OEW configuration. In various embodiments, introducing the at least one sperm into the isolation pen may include using electro-wetting force. The electro-wetting force may be produced by an OEW configuration.

In other embodiments, introducing the ovum into the isolation pen may include using fluid flow and/or gravity to transport the ovum. In some embodiments, introducing the at least one sperm into the microfluidic device may include using fluid flow and/or gravity to transport the at least one sperm.

In various embodiments, the process may further include ascertaining a status of the ovum. In some embodiments, the process may further include ascertaining a status of the ovum, where the step of ascertaining may be performed prior to introducing the at least one sperm into the microfluidic device. In some embodiments, the process may further include ascertaining a status of the ovum, wherein the step of ascertaining may be performed prior to introducing the ovum into the isolation pen.

In various embodiments of the process, at least one conditioning treatment may be performed on the ovum prior to introducing the at least one sperm into the microfluidic device. The at least one conditioning treatment may be an electrical treatment or a chemical treatment. The at least one conditioning treatment may be exposure to a somatic cell. The somatic cell may be a cumulus cell. In some embodiments, the ovum may be exposed to the somatic cell in the isolation pen.

In various embodiments of the process, the conditions conducive to fertilization of the ovum may include a composition of a medium surrounding the ovum. In various embodiments, the process may further include changing the composition of the medium surrounding the ovum prior to introducing the at least one sperm into the microfluidic device.

In other embodiments of the process, at least one conditioning treatment may be performed on the ovum after introducing the at least one sperm into the microfluidic device. The at least one conditioning treatment may be an electrical treatment or a chemical treatment.

In various embodiments, the process may further include the step of determining that the contacted ovum and the at least one sperm have formed the embryo. In some embodiments, the step of determining that the embryo has formed may include visual inspection. In some embodiments, the step of determining that the embryo has formed may include imaging. In other embodiments, the step of determining that the embryo has formed may include detecting secretions within or coming from the isolation pen in which the ovum was introduced. In some embodiments, the detection of secretions may include detecting protein or nucleic acid.

In various embodiments, the process may further include the step of performing at least one conditioning treatment on the embryo. The at least one conditioning treatment performed on the embryo may be exposure to a somatic cell. In some embodiments, the somatic cell to which the embryo is exposed may be a cumulus cell, an endometrial cell, a non-ciliated secretory cell, a PEG cell or any combination thereof. In other embodiments, the somatic cell to which the embryo is exposed is a cumulus cell and at least one cell selected from the group of an endometrial cell, a non-ciliated secretory cell, and a PEG cell.

In various embodiments of the process, each of the ovum and the at least one sperm may be obtained from a mammal.

In various embodiments of the process, the isolation pen may contain a single ovum. The microfluidic device may contain a plurality of isolation pens. In some embodiments, at least one ovum may be introduced into each of two or more isolation pens of the plurality. In other embodiments, a single ovum may be introduced into each of two or more isolation pens of the plurality. In various embodiments, the microfluidic device may further include a channel configured to contain a fluidic medium; and the isolation pen may include an isolation region and a connection region, wherein a proximal opening of the connection region fluidically connects the isolation region to the channel. The isolation region of the isolation pen may exchange components of a fluidic medium within the isolation region with components of the fluidic medium in the channel only by diffusion.

In various embodiments of the process, the process may further include the step of determining that the contacted ovum and the at least one sperm have formed the embryo; and altering a composition of a medium surrounding the embryo in the isolation pen. In some embodiments, the composition of the medium may be altered more than once as the embryo develops from a single cell embryo into a morula or a blastula. In some embodiments, altering the composition of the medium may include altering the pH of the medium.

In various embodiments of the process, the process may further include the step of exporting the embryo out of the isolation pen. In various embodiments of the process, the process may further include the step of exporting the embryo out of the microfluidic device.

In another aspect, a process is provided for monitoring a status of at least one biological micro-object in a microfluidic device, where the biological micro-object is selected from an embryo, sperm or ovum, the process including the steps of introducing the biological micro-object into an isolation pen of the microfluidic device; providing a medium to the biological micro-object; analyzing a secretion produced by the biological micro-object; and determining the status of the biological micro-object. In some embodiments, the step of providing a medium to the biological micro-object may further include providing a medium to the biological micro-object configured to provide nutrients necessary for viability.

In various embodiments of the process, the provided medium may include components necessary to activate the biological micro-object for a subsequent biological transformation. In some embodiments, the subsequent biological transformation may be fertilization or advancing to a subsequent stage of embryonic development.

In various embodiments of the process, the step of analyzing the secretion may include capturing the secretion with a capture bead. In some embodiments, the step of analyzing the secretion may be performed within or immediately adjacent to the isolation pen. In other embodiments, the step of analyzing the secretion may be performed outside of the microfluidic device. In various embodiments, the step of analyzing the secretion may include detecting proteins, nucleic acids, fragments of any of the foregoing, or any combination thereof. In some embodiments, the step of analyzing the secretion may be performed two or more times. In some embodiments, the step of analyzing the secretion may be performed periodically.

In various embodiments of the process, the process further includes the step of imaging the biological micro-object, where at least one image of the biological micro-object may be used in conjunction with at least one analysis of the secretion for determining the status of the biological micro-object.

In various embodiments of the process, the process further includes the step of exporting the biological micro-object from the isolation pen.

In various embodiments of the process, the biological micro-object is an embryo, and where the embryo may be exported after determining that the embryo is viable. In various embodiments, the biological micro-object is an embryo, and where the embryo may be exported after determining that the embryo is a viable blastula.

In another aspect, a process is provided for monitoring a status of at least one biological micro-object in a microfluidic device, where the biological micro-object is selected from an embryo, sperm or ovum, the process includes the steps of introducing the biological micro-object into an isolation pen of the microfluidic device; providing a medium to the biological micro-object; imaging the biological micro-object; and determining the status of the biological micro-object. In some embodiments, the step of providing a medium to the biological micro-object include providing a medium configured to provide nutrients necessary for viability.

In various embodiments of the process, the step of imaging the biological micro-object may be performed more than one time. In some embodiments, the step of imaging may be performed periodically. In other embodiments, the step of imaging may be performed continuously.

In various embodiments of the process, the step of determining the status may include determining a size, a shape or both of an ovum.

In various embodiments of the process, the process further includes the step of determining whether to perform a conditioning treatment on the ovum based on the determined size and/or determined shape of the ovum.

In various embodiments of the process, the process further includes the step of determining whether the ovum is prepared for fertilization based on the determined size and/or determined shape of the ovum.

In various embodiments of the process, the step of determining the status may include determining at least one of size, shape, motility and chemotactic responses of a sperm. In various embodiments of the process, the process may further include the step of determining whether to perform a conditioning treatment on the sperm based on the determined size and/or determined shape and/or determined motility and/or chemotactic response of a sperm. In some embodiments, the step of determining whether to perform a conditioning treatment on the sperm may be based on the determination of a status of at least one of size, shape, motility and chemotactic responses of a sperm.

In various embodiments of the process, the step of determining the status may include determining whether an embryo has been formed. In various other embodiments of the process, the step of determining the status may include determining at least one of a size, a shape, and a timing of cell divisions of an embryo. In some embodiments, the timing of cell divisions may be an indicator of embryo viability.

In another aspect, a method is provided for producing a parthenogenetic embryo in a microfluidic device, including the steps of introducing an oocyte into an isolation pen of the microfluidic device; and applying a stimulating agent, thereby converting the oocyte into the parthenogenetic embryo.

In various embodiments of the method, the oocyte may be a mammalian oocyte. In various embodiments, the oocyte may be a human oocyte. In various embodiments of the method, the parthenogenetic embryo may be heterozygous. In other embodiments, the parthenogenetic embryo may be homozygous.

In various embodiments of the method, the stimulating agent may be electrical stimulation, chemical stimulation, or a combination of both. In some embodiments, the stimulating agent may be electrical stimulation.

In various embodiments of the method, the method may further include the step of exporting the parthenogenetic embryo out of the isolation pen. In various embodiments of the method, the method may further include the step of exporting the parthenogenetic embryo out of the microfluidic device.

In various embodiments of the method, the method may further include the step of converting the parthenogenetic embryo into one or more embryonic stem cells (ESCs). In various embodiments of the method, the step of converting the parthenogenetic embryo into one or more embryonic stem cells may further include isolation of the inner cell mass (ICM) from a hatched blastocyst. In various embodiments of the method, the step of converting the parthenogenetic embryo into one or more embryonic stem cells may further include culturing the ICM within an isolation pen of the microfluidic device. The step of culturing the ICM within an isolation pen may further include co-culturing the ICM with feeder cells. In some embodiments, the step of co-culturing the ICM with feeder cells may include disposing the feeder cells into isolation pens adjacent to the isolation pen wherein the ICM is disposed.

In various embodiments of the method, the step of converting the parthenogenetic embryo into one or more embryonic stem cells may further include converting the ICM into one or more embryonic stem cells (ESCs). In some embodiments, the one or more ESCs may be substantially homozygous. In some embodiments, the substantially homozygous ESCs may be diploid and may be homozygous for a mutation allele. In other embodiments, the one or more ESCs may be substantially heterozygous. In some embodiments, the one or more ESCs may be human leukocyte antigen (HLA)-matched to a donor of the oocyte.

### BRIEF DESCRIPTION OF THE DRAWINGS

FIG. 1 illustrates an example of a system for use with a microfluidic device and associated control equipment according to some embodiments of the invention.

FIGS. 2A and 2B illustrate a microfluidic device according to some embodiments of the invention.

FIGS. 2C and 2D illustrate isolation pens according to some embodiments of the invention.

FIG. 2E illustrates a detailed isolation pen according to some embodiments of the invention.

FIG. 2F illustrates a microfluidic device according to an embodiment of the invention.

FIG. 3A illustrates a specific example of a system for use with a microfluidic device and associated control equipment according to some embodiments of the invention.

FIG. 3B illustrates an imaging device according to some embodiments of the invention.

FIG. 4 is a diagram of a microfluidic device having a flow path and an isolation pen in which an embryo is located.

FIG. 5A-C are diagrams of the microfluidic device of FIG. 4 having beads located in the flow path. The embryo in the isolation pen is secreting analytes, which can diffuse towards and be captured by the beads in the flow path. The beads can be analyzed with respect to amount and type of analytes bound at one or more time points during embryonic development, including the one-cell stage (FIG. 5A), the two-cell stage (FIG. 5B), the four-cell stage (FIG. 5C), or any other stage of interest.

### DETAILED DESCRIPTION OF THE INVENTION

This specification describes exemplary embodiments and applications of the invention. The invention, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least two ports configured to allow the fluid (and, optionally, micro-objects suspended in the fluid) to flow into and/or out of the microfluidic device. Typically, a microfluidic circuit of a microfluidic device will include at least one microfluidic channel and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. Typically, a nanofluidic device will comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements is configured to hold a volume of fluid of about 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A “microfluidic channel” or “flow channel” as used herein refers to a flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is in the range of from about 50,000 microns to about 500,000 microns, including any range therebetween. In some embodiments, the horizontal dimension is in the range of from about 100 microns to about 1000 microns (e.g., about 150 to about 500 microns) and the vertical dimension is in the range of from about 25 microns to about 200 microns, e.g., from about 40 to about 150 microns. It is noted that a flow channel may have a variety of different spatial configurations in a microfluidic device, and thus is not restricted to a perfectly linear element. For example, a flow channel may include one or more sections having any of the following configurations: curve, bend, spiral, incline, decline, fork (e.g., multiple different flow paths), and any combination thereof. In addition, a flow channel may have different cross-sectional areas along its path, widening and constricting to provide a desired fluid flow therein.

As used herein, the term “obstruction” refers generally to a bump or similar type of structure that is sufficiently large so as to partially (but not completely) impede movement of target micro-objects between two different regions or circuit elements in a microfluidic device. The two different regions/circuit elements can be, for example, a microfluidic isolation pen and a microfluidic channel, or a connection region and an isolation region of a microfluidic isolation pen.

As used herein, the term “constriction” refers generally to a narrowing of a width of a circuit element (or an interface between two circuit elements) in a microfluidic device. The constriction can be located, for example, at the interface between a microfluidic isolation pen and a microfluidic channel, or at the interface between an isolation region and a connection region of a microfluidic isolation pen.

As used herein, the term “transparent” refers to a material which allows visible light to pass through without substantially altering the light as is passes through.

As used herein, the term “micro-object” refers generally to any microscopic object that may be isolated and collected in accordance with the present invention. Non-limiting examples of micro-objects include: inanimate micro-objects such as microparticles; microbeads (e.g., polystyrene beads, Luminex™ beads, or the like); magnetic beads; microrods; microwires; quantum dots, and the like; biological micro-objects such as cells (e.g., embryos, oocytes, ova, sperm cells, cells dissociated from a tissue, eukaryotic cells, protist cells, animal cells, mammalian cells, human cells, immunological cells, hybridomas, cultured cells, cells from a cell line, cancer cells, infected cells, transfected and/or transformed cells, reporter cells, prokaryotic cells, and the like); biological organelles; vesicles, or complexes; synthetic vesicles; liposomes (e.g., synthetic or derived from membrane preparations); lipid nanorafts (as described in Ritchie et al. (2009) “Reconstitution of Membrane Proteins in Phospholipid Bilayer Nanodiscs,” Methods Enzymol., 464:211-231), and the like; or a combination of inanimate micro-objects and biological micro-objects (e.g., microbeads attached to cells, liposome-coated micro-beads, liposome-coated magnetic beads, or the like). Beads may further have other moieties/molecules covalently or non-covalently attached, such as fluorescent labels, proteins, small molecule signaling moieties, antigens, or chemical/biological species capable of use in an assay.

As used herein, the term “maintaining (a) cell(s)” refers to providing an environment comprising both fluidic and gaseous components and, optionally a surface, that provides the conditions necessary to keep the cells viable and/or expanding.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result.

The phrase “substantially no flow” refers to a rate of flow of a fluidic medium that, averaged over time, is less than the rate of diffusion of components of a material (e.g., an analyte of interest) into or within the fluidic medium. The rate of diffusion of components of such a material can depend on, for example, temperature, the size of the components, and the strength of interactions between the components and the fluidic medium.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a microfluidic device is an example of a swept region while an isolation region (described in further detail below) of a microfluidic device is an example of an unswept region.

As used herein, a “flow path” refers to one or more fluidically connected circuit elements (e.g. channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein: μm means micrometer, μm³ means cubic micrometer, pL means picoliter, nL means nanoliter, and μL (or uL) means microliter.

As used herein, “embryo” refers to the product of a fertilized egg at any stage of development prior to implantation. Thus, the term embryo encompasses zygote, morula, blastula and the like.

Embodiments of the invention allow monitoring the status of a biological micro-object, such as an embryo, sperm, or ovum, while the biological micro-object is located in a microfluidic (or nanofluidic) device. The monitoring can involve optical, chemical and/or electrical analyses. The monitoring can further include conditioning treatments of the biological micro-object, which may be performed prior to and/or after the optical, chemical and/or electrical analyses of the biological micro-object. The status of the biological micro-object can be determined relative to other corresponding biological micro-objects. Alternatively, the status of the biological micro-object can be determined relative to predetermined characteristics. Such predetermined characteristics can be correlated with health and viability.

In some embodiments, the biological micro-object is loaded into the microfluidic device or a particular region therein, such as an isolation pen, prior to monitoring its status. The microfluidic device may have a first region containing at least one microfluidic channel and one or more (e.g., a plurality of) isolation pens, where the pens open into the channel. Each pen may be configured to have an isolation region and a connection region, where the isolation region exchanges components of fluidic medium within the isolation region with components of the fluidic medium in the channel only by diffusion. The first region of the microfluidic device may provide the location where biological micro-objects such as oocytes, ova, or embryos, may be maintained individually, one in each isolation pen. In some embodiments, sperm may also be stored in isolation pens, but may be maintained either as a group within an isolation pen or may be maintained individually in an isolation pen.

In some embodiments, the microfluidic device may further include a second region. The second region may be a selection region and may be located upstream from the first region. The selection region may contain at least one channel which may be connected with a channel of the first region (if one exists). Optionally, the selection region can include no isolation pens (e.g., the selection region can consist or consist essentially of a channel). The length of the channel within the selection region may be the same as the length of a channel in the first region or the length of the channel in the selection region may be 1, 2, 3, 5, 7, 9, or 25 times the length of the channel in the first region. The selection region may be disposed between an inlet port and the isolation region.

The selection region may be used to select imported biological micro-objects for placement within selected isolation pens in the isolation region or for testing within the selection region itself. The extended channel in the selection region may be used to provide a swimming region for sperm introduced into the microfluidic device. The swimming region (extended channel) may select for the most motile (fit) sperm in the fertilization process. The fastest sperm will reach the isolation pens having an ovum in it before the slower, less fit sperm can reach the ovum. (See Garcia et al, U.S. Pat. No. 9,079,189, herein incorporated by reference in its entirety).

Loading of biological micro-objects or micro-objects such as, but not limited to, beads, can involve the use of fluid flow, gravity, a dielectrophoresis (DEP) force, electrowetting, a magnetic force, or any combination thereof. The DEP force can be generated optically, such as by an optoelectronic tweezers (OET) configuration and/or electrically, such as by activation of electrodes/electrode regions in a temporal/spatial pattern. Similarly, electrowetting force may be provided optically, such as by an opto-electro wetting (OEW) configuration and/or electrically, such as by activation of electrodes/electrode regions in a temporal spatial pattern.

In some embodiments, the biological micro-object is formed within the microfluidic device (e.g., the formation of an embryo by the fertilization of an ovum within the microfluidic device), prior to monitoring its status. In such embodiments, an ovum and/or a sperm can be monitored prior to fertilization and the resulting embryo can be monitored after its formation.

Monitoring.

In some embodiments, monitoring the status of the biological micro-objects includes detecting the morphology and/or movement of the biological micro-object while it is in the microfluidic device. Such detection can involve observation through microscopes, or imaging the biological micro-object one or more times (e.g., periodically) or continuously (e.g., producing video recordings). For embryos, such observation or imaging can be used to determine size, shape, and the timing of cell divisions. The time of cell divisions can be used as an indicator of embryo viability. For ova, such observation or imaging can be used to evaluate size and shape. For sperm, such observation or imaging can be used to evaluate size, shape, motility and/or chemotactic responses. For both ova and sperm, evaluation (e.g., ascertaining a status of an ovum or a sperm) made possible by the monitoring of morphology and/or movement within an isolation pen or within a channel in the selection region upstream thereof, may lead to the determination to provide conditioning treatments (which may include stimuli or other augmentative treatment) to enhance the viability and/or activity of ova or sperm. The monitoring and/or evaluation may occur at 1, 2, 3, 4, or more time points during a fertilization procedure, and may continue for 1, 2, 3, 4, or more time points after fertilization. A status of an ovum may be ascertained prior to disposing the ovum within the isolation region of an isolation pen (e.g., within the microfluidic channel) or may be ascertained when the ovum is already disposed within the isolation region of the isolation pen.

Morphology.

The ease of visualization within the microfluidic device and isolation pens described herein provide enhanced opportunity for morphological evaluation and ranking of ova, sperm and/or embryos. Visual inspection may include visual inspection using a microscope, imaging via the optical system of the instrument containing the microfluidic device, or obtaining video images, which may be projected or accessed remotely. In one non-limiting example, oocytes, ova, or embryos may be assessed and ranked for quality using a consensus assessment established by scientific working groups. Some ranking criteria may be found in “The Istanbul consensus workshop on embryo assessment: proceedings of an expert meeting”, Human Reproduction, vol. 26, No. 6. pp 1270-1283, the contents of which are incorporated by reference in their entirety. Standardized comparison may be made for cytoplasmic characteristics, pronuclear characteristics, polar body behavior (e.g., position of the second polar body), and embryo fragmentation. Additionally, morphokinetic variables may be used to determine viable embryos. Some useful comparators may be time of division to 5 cells (about 48 to about 57 h); time between division from 3 cells to 4 cells (less than about 0.76 h); and duration of the second cycle of cell division (time from division to 2 cells to division to 3 cells, less than about 12 h). (Milachich, BioMed Res. Intl. 2014, Article ID 306505). In some embodiments, the extent of fragmentation may be inversely related to quality of embryonic development, and the capability to closely investigate such fragmentation is an advantage of embryonic culture in the microfluidic devices described herein.

Non-Invasive Analysis.

In some embodiments, monitoring the status of the biological micro-object includes analyzing one or more secretions from the biological micro-object. The secretions can include proteins, nucleic acids, carbohydrates, metabolites, fragments of any of the foregoing, or any combination thereof. The analysis can include, for example, proteomic and/or genomic assessments of such secretions. In some embodiments, the secretions can be analyzed, either partially or entirely, while located within the microfluidic device. In other embodiments, the secretions can be analyzed, either partially or entirely, after being exported from the microfluidic device. For example, an aliquot of the fluid within an isolation pen can be taken for analysis of any secretions present therein. The aliquot can be combined with suitable reagents (e.g., reagents that react with secretions in the aliquot to produce a detectable signal) either within the microfluidic device or after being exported therefrom. Alternatively, or in addition, secretions from the biological micro-object(s) in an isolation pen can be captured on one or more capture beads, and the capture beads can be analyzed for secretions bound thereto either within the microfluidic device or after being exported therefrom. In some embodiments, the analysis of the secretions is repeated over time, and a time resolved secretion profile can be generated for the biological micro-object in the isolation pen.

In some embodiments, secretion analysis (e.g., single time-point or time resolved secretion profiles) and/or other information (e.g., morphological and/or motility data, either single time-point or time resolved) can be utilized to select a biological micro-object for further processing. For example, secretion analysis and/or other information can be used to select an ovum and/or a sperm for fertilization, or to select an embryo for implantation. US 2015/0151298 (application Ser. No. 14/520,568, filed Oct. 22, 2014), and US 2015/0165436 (application Ser. No. 14/521,447, filed Oct. 22, 2014), incorporated herein by reference in their entirety, describe exemplary methods of analyzing secretions of cells cultured in a microfluidic device, particularly any of microfluidic devices 100, 200, 240, 290.

Advantages of some embodiments of the invention include the ability to place biological micro-objects, such as embryos, sperm, ova, or oocytes in confined spaces while maintaining their viability, allowing detection and/or tracking of their morphology and motility, and generating concentrations of secretions that are high enough to be accurately assayed. The size of the confined space that an ovum, embryo, or sperm occupies within the isolation pens of the microfluidic device may be in the range of about 5 to about 50 times the size of a human ovum or embryo (e.g., about 2 nL to about 10 nL, about 2 nL to about 20 nL, about 5 nL to about 15 nL, about 5 nL to about 20 nL, about 5 to about 25 nL, about 10 nL to about 20 nL, about 10 nL to about 30 nL, about 10 to about 40 nL or about 10 nL to about 50 nL. Other advantages include using beads or taking small aliquots of media to measure time response of secretion; moving aliquots of media and/or beads without disturbing the local environment of the biological micro-object; and analyzing secreted or released substances (e.g., proteins, nucleic acids, carbohydrates, metabolites, and/or fragments thereof) to collect information regarding the status and quality of the biological micro-object, thereby enabling the selection of preferred (e.g., healthy, viable, etc.) biological micro-objects. Using the microfluidic devices as described herein, the ability to select just one of a plurality of biological micro-objects advantageously permits highly selective disposition within a specified location within the microfluidic device and correlation between status of the micro-object and its location. Additionally, import and export of biological micro-objects may likewise be highly selective and specific, another advantageous aspect of the methods described herein. The ease of monitoring and imaging provides further advantageous aspects of the methods. Further, the ease of importation of assay beads having specific and selective capture materials permits great flexibility in assaying the biological micro-objects non-invasively. The ability to use very small culture volumes which are well segregated from each other also permits surprisingly specific and selective ability to monitor, and optionally, advance the status of a particular biological micro-object towards greater reproductive fitness. Alternatively, the same attributes of the methods described herein may permit earlier and more precise identification of a biological micro-object that should be deselected from progression within an assisted reproduction process. All of these capabilities answer an urgent and unfilled need within the field of assisted reproduction (e.g., for human assisted reproduction) and more broadly, within the reproductive technology field.

Capture Beads.

Beads used in the analyses described herein may be made of any suitable material including glass, polymeric materials and magnetic materials. The beads may further have a coating or shell over the base material which provide a substrate for attaching capture materials such as oligonucleotides, proteins, antibodies, antigens, polysaccharides, or synthetic molecules designed to bind a biomolecule secreted or released from an ovum, sperm, or embryo. The capture materials may bind soluble or other extracellular embryonic materials such as proteins, nucleic acids, carbohydrates, metabolites, and/or fragments thereof. Detection may include detecting the entire soluble or extracellular embryonic secretion or fragments thereof.

Analyses.

A variety of non-invasive analyses may be carried out on biological micro-objects such as oocytes, ova, sperm or embryos. Advantageously, analysis performed in-vitro within the microfluidic device, eliminates the confounding effects of DNA contamination (e.g., from the mother) can be performed prior to implantation; and can be performed within a volume of media surrounding or adjacent to a single embryo of interest that is much smaller than that of typical IVF conditions. Thus the concentration of embryo-free DNA or other secreted substances may be significantly increased, thereby increasing the probability of effective test material capture from the medium surrounding the cell of interest. Analysis as described herein includes collection of the analyte within the microfluidic device. The processing of the analyte to yield a status or evaluated state of the biological micro-object may take place within the microfluidic device. Alternatively, the processing of the captured analyte may be performed outside of the microfluidic device (e.g., amplification of captured nucleic acids and subsequent detection of the amplification product).

Cell-Free DNA.

Detection of single gene defects in an oocyte, an ovum, a sperm or an ovum may be possible by capturing cell free DNA onto capture beads having capture oligonucleotides specific to a defect of interest. Sufficient DNA may be released from a single ovum or embryo to permit its capture onto capture beads either in an isolation pen containing an ova or embryo or in the microfluidic channel adjacent to the proximal opening of the isolation pen. The capture beads may have capture material bound or associated with the beads that is configured to either capture all nucleic acid or may be configured to capture one or more specific subsets of nucleic acid (e.g., gDNA, mDNA, mRNA, rRNA, miRNA, etc.). The capture material may be based on interactions such as, but not limited to, charge affinity or sequence complementarity. It has been shown that microliter volumes of exhausted cell culturing media contain sufficient cell-free DNA to be captured and subjected to analysis in order to detect alpha globin gene deletions causing alpha thalassemia via fluorescent gap polymerase chain reaction (PCR) analysis. (Wu et al., Medicine 2015; 94; e669). Other single gene defects, (e.g., Tay Sachs, BRCA, or cystic fibrosis) may be detected by design of capture oligonucleotides specific to one or more regions of the targeted gene. Capture beads bearing these binding oligonucleotides may capture sufficient DNA for detection by massively parallel sequencing (Next Generation Sequencing (NGS)), quantitative PCR (qPCR), digital PCR (dPCR), real-time PCR using dual molecular beacon reporter probes, or microarray detection. Nested PCR may be used to amplify the captured DNA sufficiently while reducing errors due to high cycle numbers. Comparative genome hybridization, using a differentially labeled control sample within the PCR reaction may decrease the effect of biased PCR.

Besides single gene defects, SNP arrays may be used for aneuploidy analysis of an oocyte, ovum, or embryo, including with low-input samples that have been efficiently amplified. dPCR may also be used for aneuploidy analysis, where primers directed to a polymorphic allele in the chromosome of interest can provide a different balance of signals for an aneuploid compared to an euploid oocyte, ovum, or embryo. Short Tandem Repeat (STR) microsatellite fragment analysis may be used to detect aneuploidy using multiplex quantitative fluorescence PCR (QF-PCR). A 10plex QF-PCR panel directed towards aneuploidies of the X and Y chromosome has been demonstrated. The panel includes two autosomal STRs. (Xie, PLOS one 2014: 9: e106307). Another panel, recently developed, is directed against aneuploidies of chromosome 13, 18, 21, X and Y using homologous gene quantitative PCR (HGQ-PCR) to obtain the same copy number information as more time consuming karyotyping assays (Long, Mol. Med. Reports: 2013:8: 1601-1605). STR analysis may be used to determine human leukocyte antigen matching, when histocompatibility for a sibling is being examined. Whole Genome Analysis (WGA) may offer the greatest amount of information, when PCR amplification error is limited. Panels focused on genetic information relevant to pre-implantation or fertilization decisions may provide critical information when used with a sample of exhausted media or secretions from the embryo obtained at a microfluidic scale.

A general measure of embryonic fitness may be made by capturing all DNA, with subsequent detection of the mtDNA/gDNA ratio. Increased presence of mtDNA may be highly correlated with fragmentation rates in the early embryo. High fragmentation rates may indicate a decreased potential for successful development and successful implantation.

Proteins.

Autocrine or paracrine secretions may be monitored via antibody capture onto beads. Increased levels of lipocalin-1 have been correlated with aneuploidy in embryos, and beads containing antibodies directed toward this protein may capture sufficient protein (e.g., as it diffuses within or from the isolation pen holding an ovum) for analysis off chip. Quantification may be enhanced if multiplex protein products are detected in this manner. Other proteins may also be analyzed for correlation with aneuploidy (from the embryo secretome), including soluble tumor necrosis factor (TNF), interleukin-10 (IL-10), macrophage-stimulating protein-alpha, (MSP-alpha); stem cell factor (SCF), chemokine (CXC-motif) ligand 13 (CXCL13), TNF-related apoptosis inducing ligand receptor 3 (TRAILR3), macrophage inflammatory protein-1beta (MIP-1beta), and GM-CSF.

Noninvasive Analysis of Physical Behavior Under an Electric Field as a Stratification Method.

In some embodiments, a dielectrophoresis field in the microfluidic device may be used to discriminate between better quality oocytes, ova, or embryos. As these cells are polarizable, the dielectrophoretic field may be used to classify the rate at which a particular cell moves under the influence of the field, in a low conductance medium (e.g., 0.3M sorbitol). In some embodiments, a more completely developed micro-object such as an oocyte, ovum, or embryo may move relatively faster than a comparatively under-developed micro-object. This may be due to differences in gene expression leading to different levels of transcription. In the microfluidic device described herein, changes of culture medium can be accomplished quickly as needed. The micro-object can be tested rapidly within a pen or in a channel, returned to a known location, and the position can be correlated with the testing result. (Garcia et al., U.S. Pat. No. 9,079,189)

Culturing in Proximity to Feeder Cells.

Embodiments of the invention also allow culturing biological micro-objects (e.g., embryos, ova, oocytes) in proximity to feeder cells that facilitate proper growth and development, that increase the likelihood of a viable pregnancy, and/or that aid in providing negative selective pressure on embryos that would not result in a viable pregnancy. Feeder cells can be located either outside or inside the microfluidic device, in a manner that allows the biological micro-objects to sample the secretions of the feeder cells. For example, when located outside of the microfluidic device, the feeder cells can be located in a chamber that media flows through prior to entering the microfluidic device. When located inside the microfluidic device, the feeder cells can be located in a region (e.g., chamber) up stream of the biological micro-objects, in a common flow path, such that the biological micro-objects sample the secretions of the feeder cells. Alternatively, the feeder cells can be located in the same isolation pen as the biological micro-objects. The feeder cells can be, for example, a population of uterine cells, endometrial cells, non-ciliated secretory cells or PEG cells derived from the uterine tube (e.g., oviduct or Fallopian tube), ovarian (cumulus cells) or a combination thereof. Cumulus cells as feeder cells may provide essential pyruvate and cysteine concentrations that oocytes, ova or embryos are incapable of metabolizing from standard culture media containing glucose and cysteine respectively. Uterine cells (or endometrial cells, non-ciliated secretory cells, and/or PEG cells) optionally in combination with cumulus cells, may provide nutrients and/or signals that support normal embryonic development. The feeder cells can be extracted, for example, from a prospective mother (e.g., biological mother or surrogate mother). Alternatively, the feeder cells could be fibroblasts or other types of cells conventionally used for supporting cell growth in vitro or ex vivo.

Culture Media.

Embodiments of the invention can also allow optimization of media during the early development of an embryo (e.g., the pre-implantation culture phase). If embryos are grown in pens in a microfluidics device, they sample the perfused medium via diffusion. Thus the medium composition can be changed in response to the monitoring of the embryos and/or the sampling of the secretions from the embryo described above. The medium composition may be altered 2, 3, 4, or more times during the period of growth of the embryo. The media composition used in the period of growth of the embryo may be altered from the medium composition used during the period of fertilization, which itself may be altered 2, 3, or more times. The composition of medium may be changed once or more as the embryo develops from a single cell embryo into a morula or a blastula. For example, different pH has been shown to be preferred at different times during embryonic development. Thus, switching between media could allow optimization of pH in response to the observed properties of the embryo.

A single medium may be used throughout the entire workflow from pre-fertilization evaluation to peri-implantation of the embryo and, optionally, may also be used in oocyte activation procedures. Non-limiting examples of a “universal” culture medium include G-TL™ (Vitrolife) and Continuous Single Culture® Complete (CSC-C, Irvine Scientific). In other embodiments, media may be designed to be sequential in nature and used during specific timeframes of oocyte/ovum/embryo development. One example of a sequential media system is the G-GAMETE™, G-1™ (pro-nucleate to d2-3), G-2™ (d3 to blastocyst) series from Vitrolife. In some embodiments, media may be designed to provide the optimized conductance for use within a microfluidic device having an OET or OEW configuration. A suitable medium may contain one or more of glucose, fructose, pyruvate, dextran, taurine, buffer (including but not limited to bicarbonate, citrate, phosphate, 4-(2-hydroxyethyl)-1-piperazineethanesulfonic acid (HEPES) or morpholinopropane-1-sulfonic acid (MOPS)), retinoic acid, hyaluronan and/or hyaluronic acid/acid salts, amino acids (all amino acids, but in some particular uses, cysteine and nonessential amino acids such as aspartate, glutamate, alanine and the like), antioxidants (including but not limited to cysteamine, vitamins (including but not limited to vitamin B related niacinamides, thiamines, pyridoxines, and/or riboflavins, E related tocopherols and tocotrienols), cytokines (including but not limited to granulocyte-macrophage colony stimulating factor (GM-CSF), antibacterial agents (e.g., gentamycin, tetracyclin) and/or chelators (one non-limiting example is ethylenediamine tetraacetic acid (EDTA)), amongst other possible components.

In some embodiments, the culture medium may include cysteine, where cysteine is present in a concentration range of about 1 micromolar to about 500 micromolar; about 10 micromolar to about 250 micromolar; about 50 micromolar to about 150 micromolar, or any value within any of these ranges. In various embodiments, the culture medium may include cysteamine, where the cysteamine is present in a concentration range of about 5 micromolar to about 1000 micromolar, about 50 micromolar to about 500 micromolar, about 100 micromolar to about 300 micromolar, or any value within any of these ranges.

In some embodiments, the medium may contain serum of the same species as the oocyte/ovum/embryo/sperm. In some embodiments, the medium may contain serum of a species different from that of the oocyte/ovum/embryo/sperm. The different species may be a different mammalian species. In other embodiments, the medium may be serum-free.

Cationic salts (including but not limited to sodium chloride, potassium chloride, magnesium sulfate, potassium phosphate, or sodium lactate) may be present in the culture medium and the conductivity may be controlled to different levels during the culturing period. The conductivity may be increased particularly during periods of culturing when dielectrophoretic force or optoelectowetting is not in use. Salt content may be decreased to provide a low conductance medium during periods of manipulation using dielectrophoretic or electrowetting. The ability to easily flow in different types of media allows for convenient changes in conductance, thus limiting exposure of biological micro-objects to low conductance medium for short periods of time.

Many different culture media are commercially available and may be suitable for use within the microfluidic device. Commercial media include, but are not limited to: G-IVF™ and G-IVF™ PLUS (Vitrolife); G-TL™ (Vitrolife); G-MOPS™ (Vitrolife); G-GAMETE™ (Vitrolife); Human Tubal Fluid (HTF), modified HTF, and complete HTF with (Serum Substitute Supplement) SSS™ (Irvine Scentific); Modified Ham's F10 or F15 Basal Medium (Irvine Scientific); Continuous Single Culture® Complete (Irvine Scientific); Multipurpose Handling Medium® Complete (Irvine Scientific); Complete Multiblast® Medium (Irvine Scientific); Complete PI® Medium (Irvine Scientific); Complete Early Cleavage® Medium with SSS™ (Irvine Scientific); Complete Early Cleavage® Medium with (Dextran Serum Supplement) DSS (Irvine Scientific); Quinn's Advantage® (Sage® Media); Global® medium (LifeGlobal® Group); G-1™ and G-2™ Series (Vitrolife); Sequential Fert™ (ORIGIO®); Sequential Fert™/Cleav™ (ORIGIO®); Sequential Cleav™/Blast™ (ORIGIO®); Sequential Blast™ (ORIGIO®); Universal IVF (ORIGIO®); BlastGen™ (ORIGIO®); ISM1™ (ORIGIO®); EmbryoGen® (ORIGIO®); BlastAssist™ (ORIGIO®); EllioStep 2 (Ellios BioMedia); BMI (Ellios BioMedia); SMART2 (Ellios BioMedia); GM501 (Gynemed); InVitroCare® HTF (InVitroCare, Inc.); InVitroCare® IVC-ONE™ (InVitroCare, Inc.); InVitroCare® IVC-TWO™ (InVitroCare, Inc.); InVitroCare® IVC-THREE™ (InVitroCare, Inc.); and Sydney IVF cleavage/blastocyst media (Cook).

Culture Medium for Activation Procedures.

A culture medium for activation may use one of the above culture media. In some embodiments, the culture medium may be Modified Ham's, G-GAMETE™; Multipurpose Handling Medium® Complete, or any of the like. In some embodiments, the medium may further have serum present. Alternatively, the medium may be serum-free. In some embodiments, the culture medium is protein-free, hypoxanthine-free, and antibiotic-free.

Dynamic Culture Conditions.

In some embodiments, a dynamic condition may be employed during some or all of the culturing period to provide gentle physical cues which can enhance embryonic development. Dynamic conditions can include one or more of tilting, perfusion, rotation, or vibration.

Isolation Pen Arrangement within the Microfluidic Device.

Embodiments of the invention can also include aligning or placing pens in close proximity with, for example, uterine or endometrial cells, optionally allowing the cells to adhere, and parking embryos in the pens. Secretions from and morphology of the embryos can then be monitored to identify the highest viability embryos. Based on evaluation, the embryo (e.g., a blastula) may be exported from the isolation pen, and out of the microfluidic device for implantation into the prospective mother. In this way, preferred embryo health and well-being can be achieved while monitoring is in progress.

Embodiments of the invention can also include sequestering individual or a group of sperm and/or ova (or oocytes) in pens, measuring their secretions and morphology, and combining sperms and ova selected based upon their respective secretions and/or morphology to form fertilized eggs. The developing embryos can then be monitored by secretion and/or morphology for suitability as described above. This could represent an integrated workflow which leverages known methods for selecting sperm, eggs, and/or embryos to improve pregnancy outcomes.

Embodiments of the invention can also provide a microenvironment for simulating the fallopian tube and/or uterine environment through time. This can be achieved, for example, by controlling the environment with the introduction of: uterine cells, endometrial cells, or cells derived from the oviduct or fallopian tube; secretions from such cell types; adjusting the pH of the media; adjusting the growth factors in the media; and/or other conditions known in the art.

Additionally, the isolation pens can be sized so as to facilitate the generation of sufficient concentrations of secretions from the biological micro-object(s) to enable accurate analysis of the secretions. Thus, for example, the isolation pen can include a volume of at least 2×10⁶ μm³, 3×10⁶ μm³, 4×10⁶ μm³, 5×10⁶ μm³, 6×10⁶ μm³, 7×10⁶ μm³, 8×10⁶ μm³, 9×10⁶ μm³, 1×10⁷ μm³, or more. The isolation pen can have a shape that is cubic or otherwise, with each of the x, y, and z dimensions being at least about as large as the diameter of the micro-object that the isolation pen is designed to hold. For example, a human ovum has a diameter of about 120 microns, so each of the x, y, z dimensions of an isolation pen designed to hold a human ovum can be at least 100, 110, 120, 130, 140, 150, or more microns.

In some embodiments, internal surfaces of the microfluidic device (e.g., internal surfaces of the isolation pens) can be conditioned so as to promote the health and viability of biological micro-objects, such as embryos, sperm, oocytes, or ova. For example, the internal surfaces can be coated with polymers, such as natural polymers (e.g., laminin, fibronectin, Matrigel, or hyaluronic acid), synthetic polymers (e.g., PEG, or PEG modified with natural polymer segments), proteins, polysaccharides, derivatives of any of the foregoing, or combinations thereof. Alternatively, or in addition, the internal surfaces can be conditioned with the secretions of support cells, such as epithelial cells or fibroblasts. Examples of such cells can include cumulus cells, endometrial cells, non-ciliary secretory cells, and PEG cells derived from the uterine tube.

Conditioning Treatments of Biological Micro-Objects.

In some embodiments, a conditioning treatment is performed to enhance fertilization success upon contacting an ovum with one or more sperm.

Electrical Treatment.

In some embodiments, one, some or all of the isolation pens may additionally be configured to provide electrical stimuli to a micro-object located therein. The electrical stimuli may be provided by modulating the DEP (OET) or electrowetting substrates at a localized position. Alternatively, the pens may have 2D planar electrodes or 3D electrodes, which may be fabricated via photolithography, or may have wire type electrodes (platinum, silver/silver chloride, and the like). In some embodiments, when the microfluidic device has a base substrate having a DEP (OET) or electrowetting (OEW) configuration and, typically, an ITO upper electrode, voltage is applied to the lower substrate. The substrate switches to a low resistance state as soon as it is illuminated with light. The medium in the isolation pen may be configured to have a conductance of about 0.01 S/M, which is higher than that of the illuminated substrate, and most of the applied field drops across the medium filling the pen. When the height of the chamber within the isolation pen is from about 30 to about 150 microns, the electrical field needed to microporate an ovum may be in about the range of about 0.1 to about 5.0 kV/cm, about 0.1, 0.3.-0.5, 0.7, 0.9, 1.0, 1.2, 1.4, 1.6 1.8, 2.0. 2.4, 2.6, 2.8, 3.0, 3.3, 3.5, 3.7, 4.0, 4.3, 4.5, 4.7 or about 5.0 kV/cm or any value in the range. In some embodiments, the electric field may be about 1.4 kV/cm. The potential required to be applied across the two electrodes will vary with the height of the chamber. The culture medium used during electrical activation may be different from the culture medium used for culturing oocytes, ova, or embryos.

Electrical stimulation may be provided in various embodiments. In one embodiment, electrical stimulation may be provided after an ovum is exposed to sperm. For example, the ovum may not be activated by the penetration of the sperm and thus may not be able to proceed with embryonic development. The sperm may have an inability to induce the intracellular calcium concentration increase necessary to promote embryo development. Application of an electrical impulse may induce the intracellular calcium ion concentration rise necessary to initiate fertilized ovum development. In some embodiments, ionophores may also be present during or after the electrical stimulus. The microporation caused by electrical impulse permits the passage of the ionophores and/or calcium ion to replace/induce the calcium (Ca⁺²) transients induced by fertilization by normally functioning sperm.

In other embodiments, electrical stimulation may be provided in the absence of sperm to initiate parthenogenetic development of an oocyte. These artificially triggered cells resume meiosis without undergoing cell division. This may be performed in the presence of ionophores such as ionomycin and/or calcium 2⁺ ions, or may be stimulated with electrical stimulation only. This may be performed in a medium specifically designed for electrofusion. For example, when DEP (e.g., OET) or an electrowetting (e.g., OEW) configuration is present within the substrate of the microfluidic device, the conductivity of an electroporation medium may be about 0.01 S/M or may be in the range of about 0.001 to 1 S/M. When human oocytes are induced to parthenogenesis, development does not proceed to full term. However, parthenogenetic human embryos may develop past the morula stage and achieve blastocyst status (32-64 cells, d4-5). The parthenogenetic blastocyst may be used to establish a human embryonic stem cell line. The blastocyst may be hatched, and the inner cell mass (ICM) may be isolated and co-cultured with appropriate feeder cells (e.g., spleen cells, fibroblasts, which may be performed sequentially). After co-culturing, primary embryonic stem cell colonies may be established. The hESC cells may be dissociated and cultured.

The steps of conversion from the parthenogenetic blastocyst to embryonic stem cell may be performed within the microfluidic device. In other embodiments, the parthenogenetic blastocyst may be exported from the microfluidic device and the remainder of the steps performed in other instrumentation. In yet other embodiments, after the ICM is isolated off-chip from the hatched blastocyst, the co-culture of the ICM with feeder cells may be performed on the same or similar microfluidic chip that stimulation was performed on. The ICM may be disposed within an isolation region of an isolation pen, and feeder cells may be co-cultured in either the same isolation pen or in adjacent isolation pens.

After conversion to embryonic stem cell colonies, the hESCs may be exported from the microfluidic device for further expansion, preservation or use.

The same steps are used for parthenogenetic embryos from non-human oocytes, in order to establish embryonic cells lines in other species, including but not limited to other mammals (e.g., mice).

Depending on the timing of stimulation with respect to the oocyte developmental stage, the parthenogenetic hESCs may be substantially homozygous or substantially heterozygous, and may typically be diploid. Stimulation of the oocyte while blocking meiosis at metaphase I stage, may provide a substantially heterozygous hESC. Stimulation while blocking meiosis after the extrusion of the second polar body, may provide a substantially homozygous hESC. Simulation at stages of oocyte development in between metaphase 1 and before extrusion of the second polar body may provide a mixture of heterozygous and homozygous hESCs.

In some embodiments, parthenogenesis of unfertilized human oocytes may be used to generate pluripotent stem cells. In some embodiments, the hESC may be HLA-matched (human leukocyte antigen) for the oocyte donor. Alternatively, electrically stimulated parthenogenetically derived hESC may establish diseased hESC cell lines without gene manipulation, yielding diploid homozygous mutation-bearing hESCs.

Chemical Treatment.

In some embodiments, an ovum or an oocyte may be treated with a chemical agent to promote the probability of success during the fertilization step. The chemical agent may be a small molecule agent or may be a biomolecule agent. In some embodiments, a chemical agent such as, but not limited to, ionomycin, calcimycin, strontium chloride, and/or calcium chloride, may be added to the culture medium of the ovum or oocyte prior to exposure to sperm.

In some embodiments, it may be possible to return normal function to non-fertile sperm by exposing the sperm to phospholipase C zeta to restore the sperm's ability to induce the Ca⁺² transients necessary to initiate normal embryonic development. Sperm may be so treated in isolation pens segregated from the ova present in other isolation pens or sperm may be treated off chip. Motility in sperm may be increased by treatment with pentoxifyline, a phosphodiesterase inhibitor, which inhibits the breakdown of cyclic adenosine monophosphate, known to be involved in sperm motility. In some embodiments, DEP forces generated by OET may be used to effect sperm penetration into an ovum, when the sperm do not have sufficiency motility or penetrability.

Exposure to a Somatic Cell.

In some embodiments, a conditioning treatment may include exposure to a somatic cell, which may include but is not limited to uterine cells, endometrial cells, cumulus granulosa cells, intercalary PEG cells, and non-ciliary secretory cells of the fallopian tubes any of which may produce enhanced concentrations of potassium, bicarbonate, arginine, alanine and glutamate, and/or prostaglandins in physiologically relevant and developmentally relevant concentrations. An ovum or an oocyte may be exposed to a cumulus granulosa cell prior to introduction of sperm. After an embryo has been formed from an ovum and sperm, a conditioning treatment may include exposure to a cumulus granulosa cell, a uterine cell, an endometrial cell, an intercalary PEG cell, a non-ciliary non-secretory cell, or any combination thereof. In some embodiments, an embryo may be subjected to a conditioning treatment which may include exposure to a cumulus granulosa cell, and one of the group consisting of an endometrial cell, an intercalary PEG cell, and a non-ciliary non-secretory cell. Exposure to somatic cells may be direct (e.g., within the same isolation pen) or indirect (e.g., in an adjacent or nearby isolation pen), where secretions from the somatic cell may enter the isolation pen containing the ovum, oocyte, or embryo by diffusion.

Rescue Activation.

In some embodiments, an initial attempt at fertilization may be performed on an ovum, and monitoring via imaging and/or testing may show that embryonic development has not progressed. In such cases, some or all of the above conditioning treatments may be employed to rescue activation to start the second meiotic division, form pronuclei and progress to normal development of an embryo.

In-Vitro Activation and In-Vitro Maturation.

In some embodiments, the conditioning treatment is performed to activate an oocyte/ovum or sperm for a subsequent biological transformation. For example, an oocyte or ovum introduced into the microfluidic device, may be monitored and tested as described herein, and found to be not developed sufficiently to have a reasonable chance of being fertilized.

As described above, sperm may be treated with agents such as, but not limited to, phospholipase C zeta or pentoxifyline to activate sperm for fertilization.

A conditioning treatment may be performed to advance the oocyte or ovum to a more matured state, more receptive to fertilization with enhanced embryonic development potential. Some non-limiting examples of conditioning treatments that may advance an oocyte or ovum to a more mature status (metaphase I, metaphase II) include human chorionic gonadotropin (hCG), follicle-stimulating hormone (FSH), retinoids (including retinoic acid), epidermal growth factor (EGF), estradiol 17beta (E2), follicular fluid meiosis-activating sterol (4,4-dimethyl-5 alpha-cholest-8,14,24-trien-3 beta-ol), brain-derived neurotropic factor, insulin-like growth factor-1, melatonin, phospholipase C zeta, and/or lysophosphatidic acid (LPA). In some embodiments, exposure of the oocyte or ovum to the conditioning chemical agent may be performed in the presence of cumulus granulosa cells.

Co-Culturing.

In some embodiments, in-vitro maturation of oocytes may be performed by co-culturing an oocyte with uterine cells, endometrial cells, cumulus granulosa cells, intercalary PEG cells, non-ciliary secretory cells of the fallopian tubes, or a combination thereof.

Microfluidic Devices and Systems for Operating and Observing Such Devices.

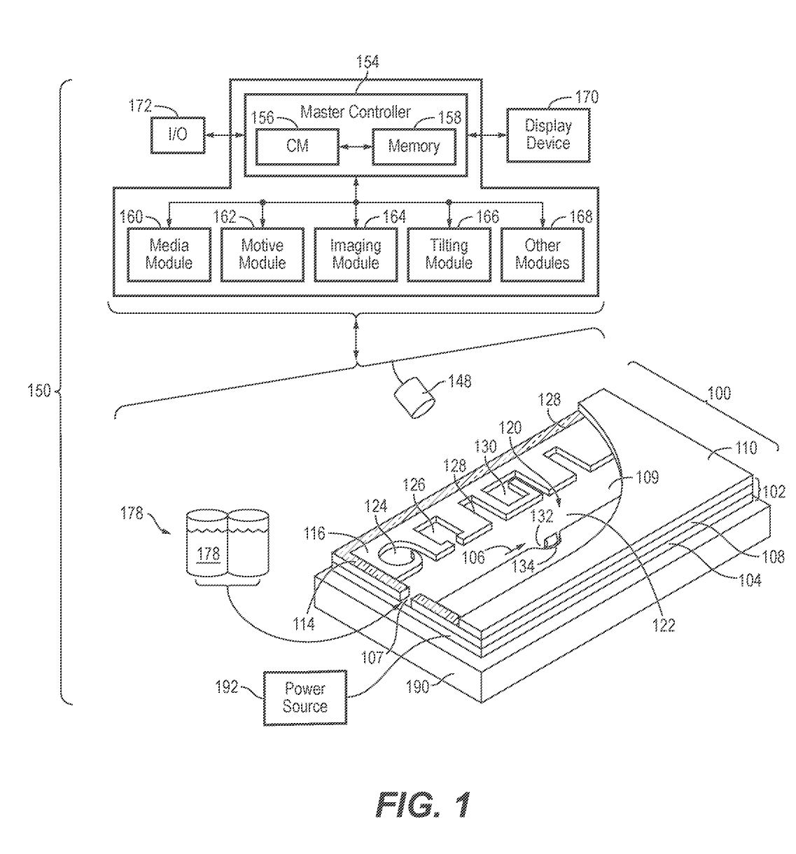

FIG. 1 illustrates an example of a microfluidic and a system 150 which can be used for generation of embryos in vitro, including selecting and evaluating ova and/or oocytes and/or sperm. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120. Although a single microfluidic circuit 120 is illustrated in FIG. 1, suitable microfluidic devices can include a plurality (e.g., 2 or 3) of such microfluidic circuits. Regardless, the microfluidic device 100 can be configured to be a nanofluidic device. In the embodiment illustrated in FIG. 1, the microfluidic circuit 120 comprises a plurality of microfluidic isolation pens 124, 126, 128, and 130, each having one or more openings in fluidic communication with flow path 106. As discussed further below, the microfluidic isolation pens comprise various features and structures that have been optimized for retaining micro-objects in the microfluidic device, such as microfluidic device 100, even when a medium 180 is flowing through the flow path 106. Before turning to the foregoing, however, a brief description of microfluidic device 100 and system 150 is provided.

As generally illustrated in FIG. 1, the microfluidic circuit 120 is defined by an enclosure 102. Although the enclosure 102 can be physically structured in different configurations, in the example shown in FIG. 1 the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120.

The support structure 104 can be at the bottom and the cover 110 at the top of the microfluidic circuit 120 as illustrated in FIG. 1. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1 but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow channels, chambers, pens, traps, and the like. In the microfluidic circuit 120 illustrated in FIG. 1, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material.

The microfluidic circuit material 116 can be patterned with cavities or the like to define circuit elements and interconnections of the microfluidic circuit 120. The microfluidic circuit material 116 can comprise a flexible material, such as a flexible polymer (e.g. rubber, plastic, elastomer, silicone, polydimethylsiloxane (“PDMS”), or the like), which can be gas permeable. Other examples of materials that can compose microfluidic circuit material 116 include molded glass, an etchable material such as silicone (e.g. photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The cover 110 can be an integral part of the frame 114 and/or the microfluidic circuit material 116. Alternatively, the cover 110 can be a structurally distinct element, as illustrated in FIG. 1. The cover 110 can comprise the same or different materials than the frame 114 and/or the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1 or integral portions of the same structure.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over isolation pens 124, 126, 128, 130) can comprise a deformable material that interfaces with rigid materials of the cover 110. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. 2012/0325665 (Chiou et al.), the contents of which are incorporated herein by reference. In some embodiments, the cover 110 can be modified (e.g., by conditioning all or part of a surface that faces inward toward the microfluidic circuit 120) to support cell adhesion, viability and/or growth. The modification may include a coating of a synthetic or natural polymer. In some embodiments, the cover 110 and/or the support structure 104 can be transparent to light. The cover 110 may also include at least one material that is gas permeable (e.g., PDMS or PPS).

FIG. 1 also shows a system 150 for operating and controlling microfluidic devices, such as microfluidic device 100. System 150, as illustrated, includes an electrical power source 192, an imaging device 194, and a tilting device 190.

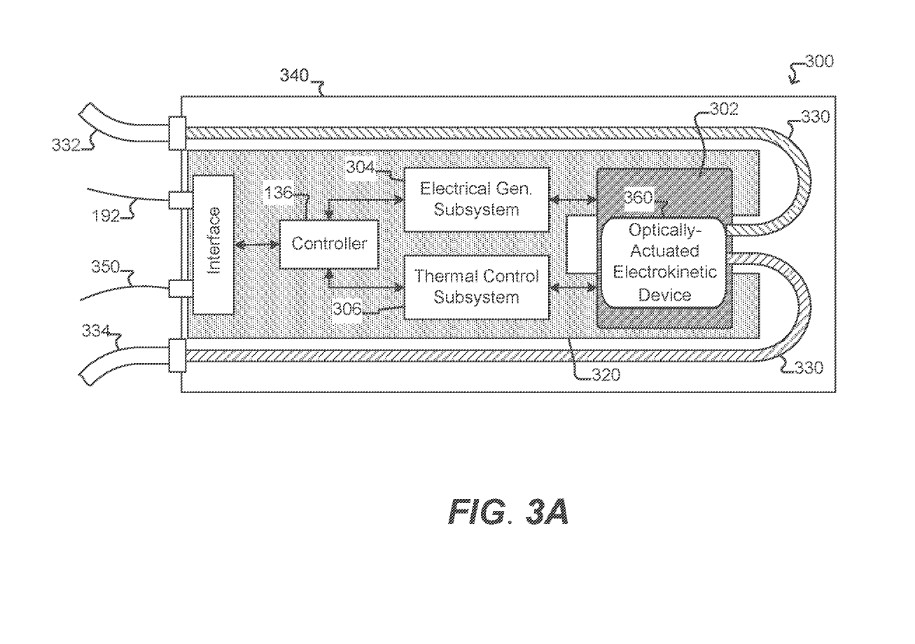

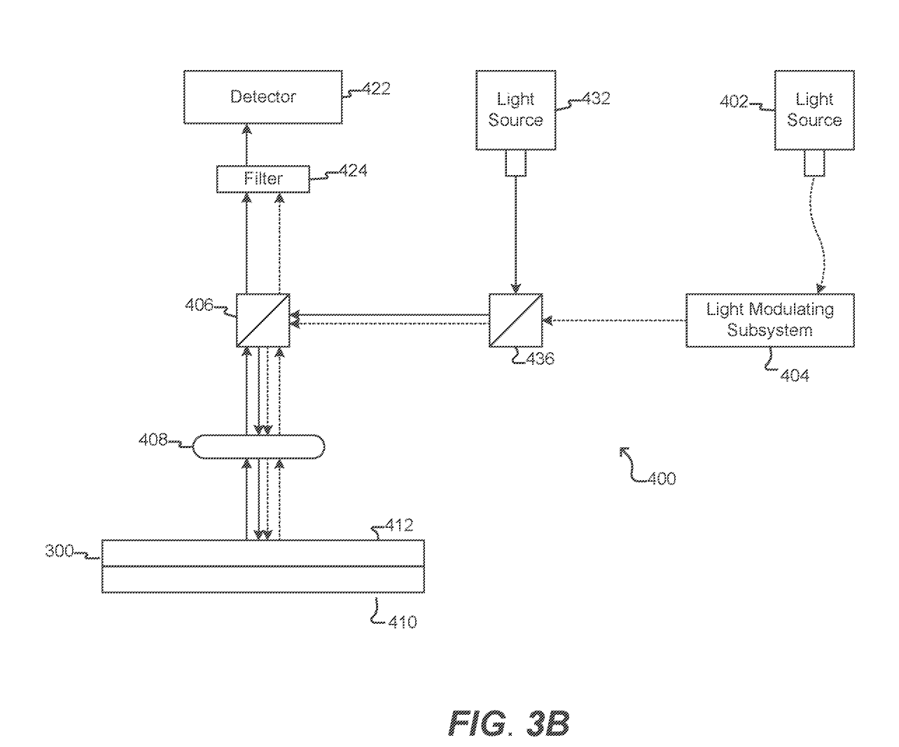

The electrical power source 192 can provide electric power to the microfluidic device 100 and/or tilting device 190, providing biasing voltages or currents as needed. The electrical power source 192 can, for example, comprise one or more alternating current (AC) and/or direct current (DC) voltage or current sources. The imaging device 194 can comprise a device, such as a digital camera, for capturing images inside microfluidic circuit 120. In some instances, the imaging device 194 further comprises a detector having a fast frame rate and/or high sensitivity (e.g. for low light applications). The imaging device 194 can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g. a high pressure mercury lamp) or a Xenon arc lamp. As discussed with respect to FIG. 3, the imaging device 194 may further include a microscope (or an optical train), which may or may not include an eyepiece.

System 150 further comprises a tilting device 190 configured to rotate a microfluidic device 100 about one or more axes of rotation. In some embodiments, the tilting device 190 is configured to support and/or hold the enclosure 102 comprising the microfluidic circuit 120 about at least one axis such that the microfluidic device 100 (and thus the microfluidic circuit 120) can be held in a level orientation (i.e. at 0° relative to x- and y-axes), a vertical orientation (i.e. at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, the tilting device 190 can tilt the microfluidic device 100 at 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 0° relative to the x-axis or any degree therebetween. The level orientation (and thus the x- and y-axes) is defined as normal to a vertical axis defined by the force of gravity. The tilting device can also tilt the microfluidic device 100 (and the microfluidic circuit 120) to any degree greater than 90° relative to the x-axis and/or y-axis, or tilt the microfluidic device 100 (and the microfluidic circuit 120) 180° relative to the x-axis or the y-axis in order to fully invert the microfluidic device 100 (and the microfluidic circuit 120). Similarly, in some embodiments, the tilting device 190 tilts the microfluidic device 100 (and the microfluidic circuit 120) about an axis of rotation defined by flow path 106 or some other portion of microfluidic circuit 120.

In some instances, the microfluidic device 100 is tilted into a vertical orientation such that the flow path 106 is positioned above or below one or more isolation pens. The term “above” as used herein denotes that the flow path 106 is positioned higher than the one or more isolation pens on a vertical axis defined by the force of gravity (i.e. an object in an isolation pen above a flow path 106 would have a higher gravitational potential energy than an object in the flow path). The term “below” as used herein denotes that the flow path 106 is positioned lower than the one or more isolation pens on a vertical axis defined by the force of gravity (i.e. an object in an isolation pen below a flow path 106 would have a lower gravitational potential energy than an object in the flow path).

In some instances, the tilting device 190 tilts the microfluidic device 100 about an axis that is parallel to the flow path 106. Moreover, the microfluidic device 100 can be tilted to an angle of less than 90° such that the flow path 106 is located above or below one or more isolation pens without being located directly above or below the isolation pens. In other instances, the tilting device 190 tilts the microfluidic device 100 about an axis perpendicular to the flow path 106. In still other instances, the tilting device 190 tilts the microfluidic device 100 about an axis that is neither parallel nor perpendicular to the flow path 106.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1. Alternatively, the media source 178 can be located in whole or in part inside the enclosure 102 of the microfluidic device 100. For example, the media source 178 can comprise reservoirs that are part of the microfluidic device 100.

FIG. 1 also illustrates simplified block diagram depictions of examples of control and monitoring equipment 152 that constitute part of system 150 and can be utilized in conjunction with a microfluidic device 100. As shown, examples of such control and monitoring equipment 152 include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device 194 (e.g., a camera, microscope, light source or any combination thereof) for capturing images (e.g., digital images), and a tilting module 166 for controlling a tilting device 190. The control equipment 152 can also include other modules 168 for controlling, monitoring, or performing other functions with respect to the microfluidic device 100. As shown, the equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, tilting module 166, and/or other modules 168 configured as discussed above. Similarly, the master controller 154, media module 160, motive module 162, imaging module 164, tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. For example, in some embodiments media module 160 stops the flow of media 180 in the flow path 106 and through the enclosure 102 prior to the tilting module 166 causing the tilting device 190 to tilt the microfluidic device 100 to a desired angle of incline.

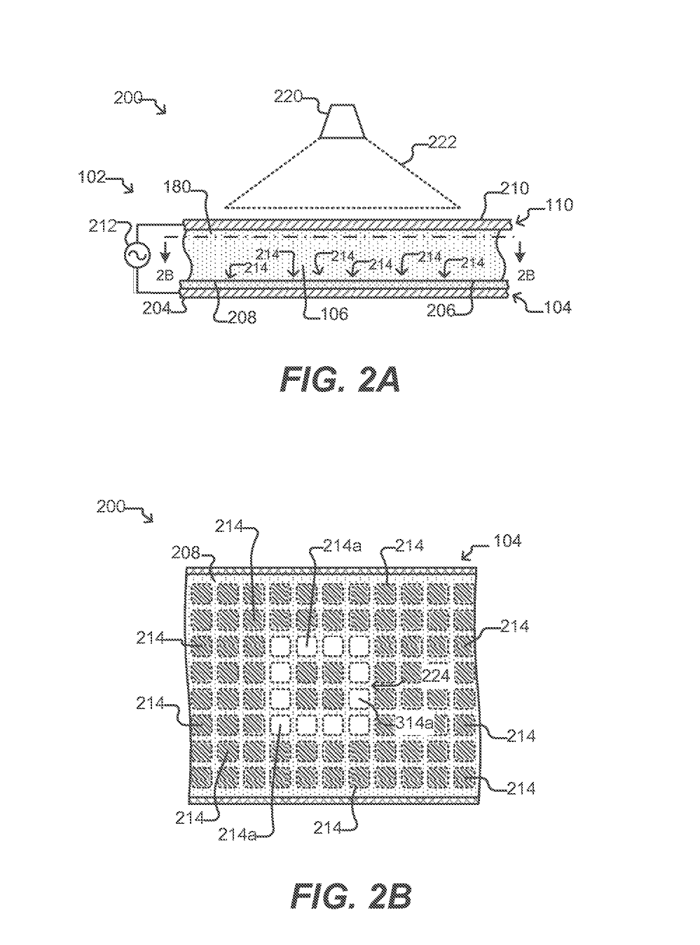

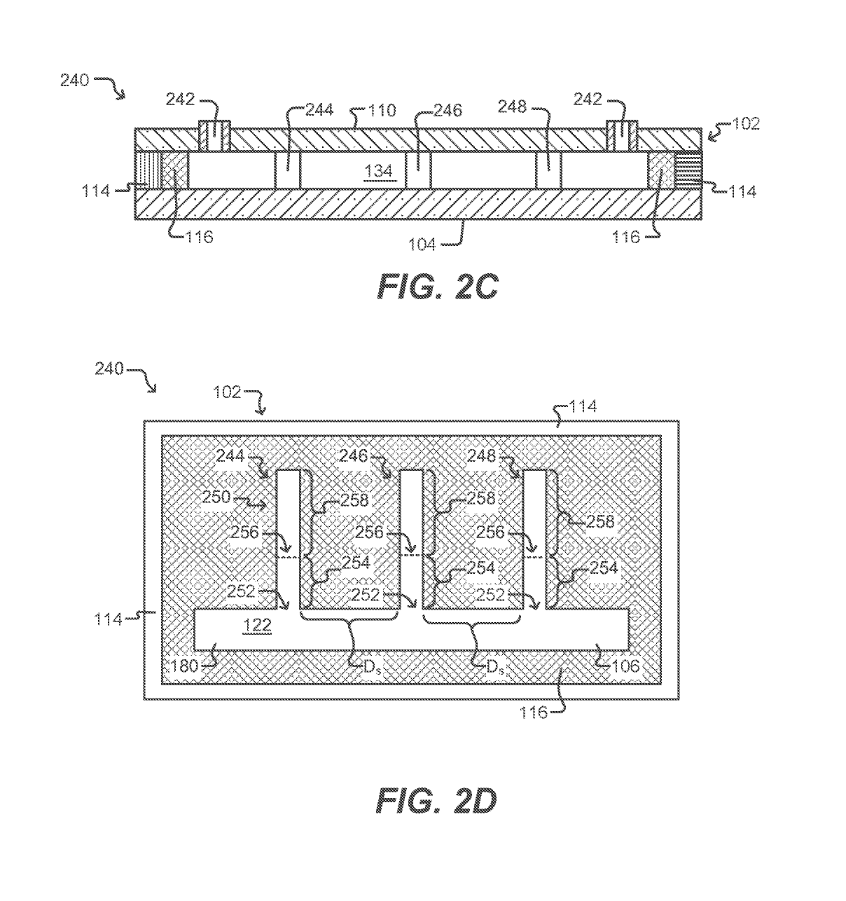

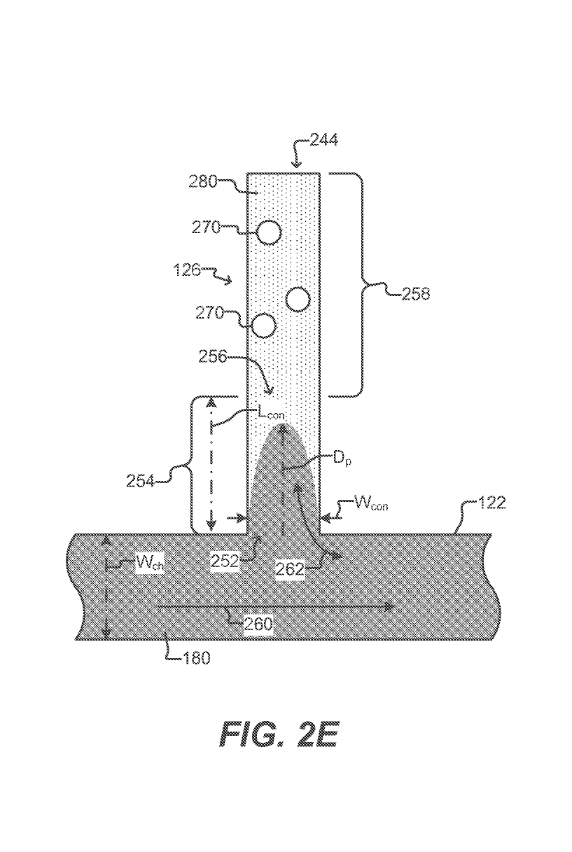

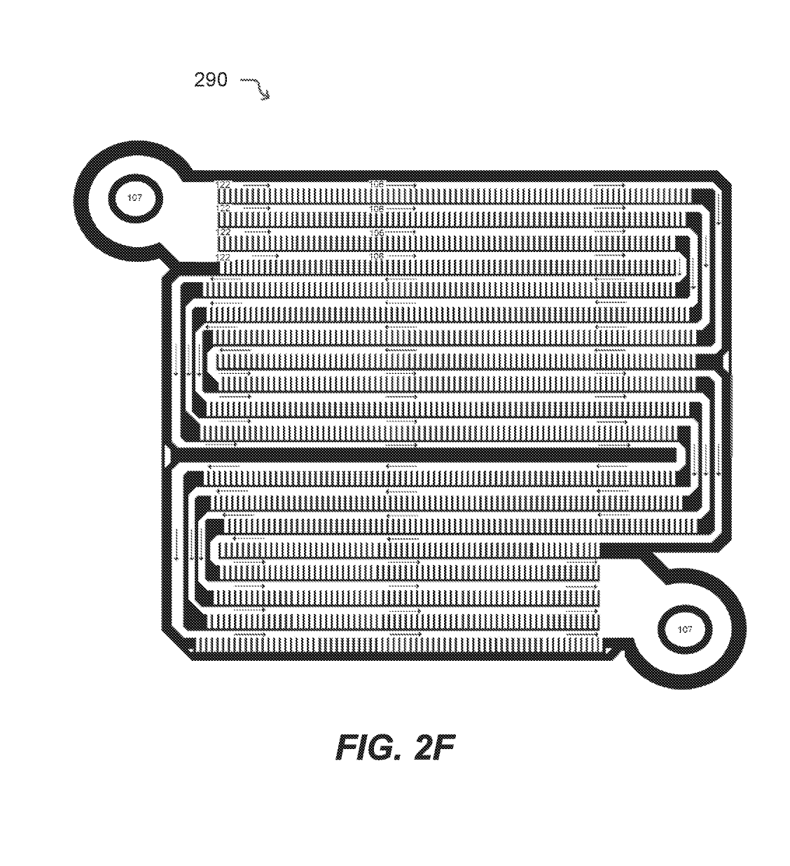

The motive module 162 can be configured to control selection, trapping, and movement of micro-objects (not shown) in the microfluidic circuit 120. As discussed below with respect to FIGS. 2A and 2B, the enclosure 102 can comprise a dielectrophoresis (DEP), optoelectronic tweezers (OET) and/or opto-electrowetting (OEW) configuration (not shown in FIG. 1), and the motive module 162 can control the activation of electrodes and/or transistors (e.g., phototransistors) to select and move micro-objects (not shown) and/or droplets of medium (not shown) in the flow path 106 and/or isolation pens 124, 126, 128, 130.

The imaging module 164 can control the imaging device 194. For example, the imaging module 164 can receive and process image data from the imaging device 194. Image data from the imaging device 194 can comprise any type of information captured by the imaging device 194 (e.g., the presence or absence of micro-objects, droplets of medium, accumulation of label, such as fluorescent label, etc.). Using the information captured by the imaging device 194, the imaging module 164 can further calculate the position of objects (e.g., micro-objects, droplets of medium) and/or the rate of motion of such objects within the microfluidic device 100.

The tilting module 166 can control the tilting motions of tilting device 190. Alternatively, or in addition, the tilting module 166 can control the tilting rate and timing to optimize transfer of micro-objects to the one or more isolation pens via gravitational forces. The tilting module 166 is communicatively coupled with the imaging module 164 to receive data describing the motion of micro-objects and/or droplets of medium in the microfluidic circuit 120. Using this data, the tilting module 166 may adjust the tilt of the microfluidic circuit 120 in order to adjust the rate at which micro-objects and/or droplets of medium move in the microfluidic circuit 120. The tilting module 166 may also use this data to iteratively adjust the position of a micro-object and/or droplet of medium in the microfluidic circuit 120.

In the example shown in FIG. 1, the microfluidic circuit 120 is illustrated as comprising a microfluidic channel 122 and isolation pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. In some instances, pens 124, 126, 128, 130 are configured to physically corral one or more micro-objects within the microfluidic circuit 120. Isolation pens in accordance with the present invention can comprise various shapes, surfaces and features that are optimized for use with DEP, OET, OEW, fluid flow, and/or gravitational forces, as will be discussed and shown in detail below.

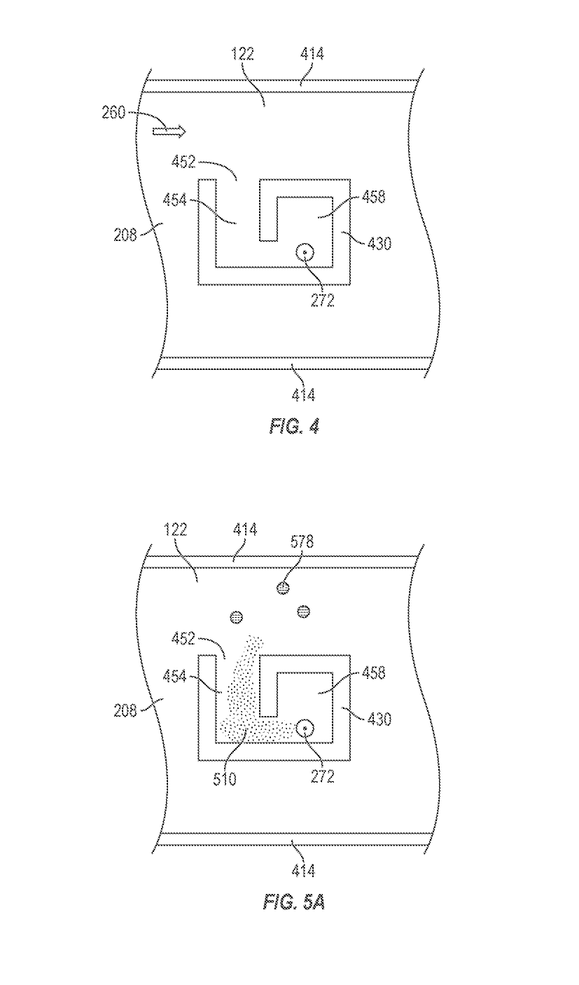

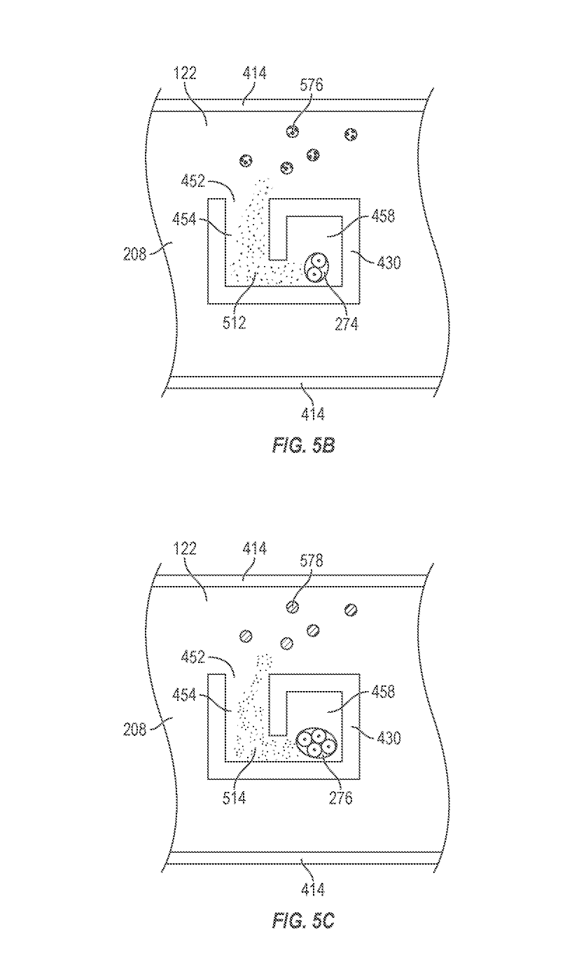

The microfluidic circuit 120 may comprise any number of microfluidic isolation pens. Although five isolation pens are shown, microfluidic circuit 120 may have fewer or more isolation pens. As shown, microfluidic isolation pens 124, 126, 128, and 130 of microfluidic circuit 120 each comprise differing features and shapes which may provide one or more benefits useful in producing an embryo, such as isolating one ovum from an adjacent ovum. Testing, stimulating and fertilizing may all be performed on an individual basis and, in some embodiments, may be performed on an individual time scale. In some embodiments, the microfluidic circuit 120 comprises a plurality of identical microfluidic isolation pens. In some embodiments, the microfluidic circuit 120 comprises a plurality of microfluidic isolation pens, wherein two or more of the isolation pens comprise differing structures and/or features which provide differing benefits in producing embryos. One non-limiting example may include maintaining ova in one type of pen while maintaining sperm in a different type of pen. In another embodiment, at least one of the isolation pens is configured to have electrical contacts suitable for providing electrical activation for an ovum. In yet another embodiment, differing types of cells (such as, for example, uterine cells, endometrial cells, PEG (intercalary) cells derived from the uterine tube (e.g., oviduct or Fallopian tube), cumulus cells, or a combination thereof) may be disposed in isolation pens adjacent to an isolation pen containing an ovum, such that secretions from the surrounding isolation pens may diffuse out of each respective pen and into the pen containing an ovum, which is not possible with macroscale in-vitro culturing and fertilization. Microfluidic devices useful for producing an embryo may include any of the isolation pens 124, 126, 128, and 130 or variations thereof, or may include pens configured like pens 430 as shown in FIGS. 4 and 5A-C, or thereof.

In the embodiment illustrated in FIG. 1, a single channel 122 and flow path 106 is shown. However, other embodiments may contain multiple channels 122, each configured to comprise a flow path 106. The microfluidic circuit 120 further comprises an inlet valve or port 107 in fluid communication with the flow path 106 and fluidic medium 180, whereby fluidic medium 180 can access channel 122 via the inlet port 107. In some instances, the flow path 106 comprises a single path. In some instances, the single path is arranged in a zigzag pattern whereby the flow path 106 travels across the microfluidic device 100 two or more times in alternating directions.

In some instances, microfluidic circuit 120 comprises a plurality of parallel channels 122 and flow paths 106, wherein the fluidic medium 180 within each flow path 106 flows in the same direction. In some instances, the fluidic medium within each flow path 106 flows in at least one of a forward or reverse direction. In some instances, a plurality of isolation pens is configured (e.g., relative to a channel 122) such that the isolation pens can be loaded with target micro-objects in parallel.

In some embodiments, microfluidic circuit 120 further comprises one or more micro-object traps 132. The traps 132 are generally formed in a wall forming the boundary of a channel 122, and may be positioned opposite an opening of one or more of the microfluidic isolation pens 124, 126, 128, 130. In some embodiments, the traps 132 are configured to receive or capture a single micro-object from the flow path 106. In some embodiments, the traps 132 are configured to receive or capture a plurality of micro-objects from the flow path 106. In some instances, the traps 132 comprise a volume approximately equal to the volume of a single target micro-object.

The traps 132 may further comprise an opening which is configured to assist the flow of targeted micro-objects into the traps 132. In some instances, the traps 132 comprise an opening having a height and width that is approximately equal to the dimensions of a single target micro-object, whereby larger micro-objects are prevented from entering into the micro-object trap. The traps 132 may further comprise other features configured to assist in retention of targeted micro-objects within the trap 132. In some instances, the trap 132 is aligned with and situated on the opposite side of a channel 122 relative to the opening of a microfluidic isolation pen, such that upon tilting the microfluidic device 100 about an axis parallel to the channel 122, the trapped micro-object exits the trap 132 at a trajectory that causes the micro-object to fall into the opening of the isolation pen. In some instances, the trap 132 comprises a side passage 134 that is smaller than the target micro-object in order to facilitate flow through the trap 132 and thereby increase the likelihood of capturing a micro-object in the trap 132.

In some embodiments, dielectrophoretic (DEP) forces are applied across the fluidic medium 180 (e.g., in the flow path and/or in the isolation pens) via one or more electrodes (not shown) to manipulate, transport, separate and sort micro-objects located therein. For example, in some embodiments, DEP forces are applied to one or more portions of microfluidic circuit 120 in order to transfer a single micro-object from the flow path 106 into a desired microfluidic isolation pen. In some embodiments, DEP forces are used to prevent a micro-object within an isolation pen (e.g., isolation pen 124, 126, 128, or 130) from being displaced therefrom. Further, in some embodiments, DEP forces are used to selectively remove a micro-object from an isolation pen that was previously collected in accordance with the teachings of the instant invention. In some embodiments, the DEP forces comprise optoelectronic tweezer (OET) forces.

In other embodiments, optoelectrowetting (OEW) forces are applied to one or more positions in the support structure 104 (and/or the cover 110) of the microfluidic device 100 (e.g., positions helping to define the flow path and/or the isolation pens) via one or more electrodes (not shown) to manipulate, transport, separate and sort droplets located in the microfluidic circuit 120. For example, in some embodiments, OEW forces are applied to one or more positions in the support structure 104 (and/or the cover 110) in order to transfer a single droplet from the flow path 106 into a desired microfluidic isolation pen. In some embodiments, OEW forces are used to prevent a droplet within an isolation pen (e.g., isolation pen 124, 126, 128, or 130) from being displaced therefrom. Further, in some embodiments, OEW forces are used to selectively remove a droplet from an isolation pen that was previously collected in accordance with the teachings of the instant invention.

In some embodiments, DEP and/or OEW forces are combined with other forces, such as flow and/or gravitational force, so as to manipulate, transport, separate and sort micro-objects and/or droplets within the microfluidic circuit 120. For example, the enclosure 102 can be tilted (e.g., by tilting device 190) to position the flow path 106 and micro-objects located therein above the microfluidic isolation pens, and the force of gravity can transport the micro-objects and/or droplets into the pens. In some embodiments, the DEP and/or OEW forces can be applied prior to the other forces. In other embodiments, the DEP and/or OEW forces can be applied after the other forces. In still other instances, the DEP and/or OEW forces can be applied at the same time as the other forces or in an alternating manner with the other forces.

FIGS. 2A-2F illustrates various embodiments of microfluidic devices that can be used in the practice of the present invention. FIG. 2A depicts an embodiment in which the microfluidic device 200 is configured as an optically-actuated electrokinetic device. A variety of optically-actuated electrokinetic devices are known in the art, including devices having an optoelectronic tweezer (OET) configuration and devices having an opto-electrowetting (OEW) configuration. Examples of suitable OET configurations are illustrated in the following U.S. patent documents, each of which is incorporated herein by reference in its entirety: U.S. Pat. No. RE 44,711 (Wu et al.) (originally issued as U.S. Pat. No. 7,612,355); and U.S. Pat. No. 7,956,339 (Ohta et al.). Examples of OEW configurations are illustrated in U.S. Pat. No. 6,958,132 (Chiou et al.) and U.S. Patent Application Publication No. 2012/0024708 (Chiou et al.), both of which are incorporated by reference herein in their entirety. Yet another example of an optically-actuated electrokinetic device includes a combined OET/OEW configuration, examples of which are shown in U.S. Patent Publication Nos. 20150306598 (Khandros et al.) and 20150306599 (Khandros et al.) and their corresponding PCT Publications WO2015/164846 and WO2015/164847, all of which are incorporated herein by reference in their entirety.

Examples of microfluidic devices having pens in which oocytes, ova, or embryos can be placed, cultured, and/or monitored have been described, for example, in US 2014/0116881 (application Ser. No. 14/060,117, filed Oct. 22, 2013), US 2015/0151298 (application Ser. No. 14/520,568, filed Oct. 22, 2014), and US 2015/0165436 (application Ser. No. 14/521,447, filed Oct. 22, 2014), each of which is incorporated herein by reference in its entirety. U.S. application Ser. Nos. 14/520,568 and 14/521,447 also describe exemplary methods of analyzing secretions of cells cultured in a microfluidic device. Each of the foregoing applications further describes microfluidic devices configured to produce dielectrophoretic (DEP) forces, such as optoelectronic tweezers (OET) or configured to provide opto-electro wetting (OEW). For example, the optoelectronic tweezers device illustrated in FIG. 2 of US 2014/0116881 is an example of a device that can be utilized in embodiments of the present invention to select and move an individual biological micro-object or a group of biological micro-objects.

Microfluidic Device Motive Configurations.

As described above, the control and monitoring equipment of the system can comprise a motive module for selecting and moving objects, such as micro-objects or droplets, in the microfluidic circuit of a microfluidic device. The microfluidic device can have a variety of motive configurations, depending upon the type of object being moved and other considerations. For example, a dielectrophoresis (DEP) configuration can be utilized to select and move micro-objects in the microfluidic circuit. Thus, the support structure 104 and/or cover 110 of the microfluidic device 100 can comprise a DEP configuration for selectively inducing DEP forces on micro-objects in a fluidic medium 180 in the microfluidic circuit 120 and thereby select, capture, and/or move individual micro-objects or groups of micro-objects. Alternatively, the support structure 104 and/or cover 110 of the microfluidic device 100 can comprise an electrowetting (EW) configuration for selectively inducing EW forces on droplets in a fluidic medium 180 in the microfluidic circuit 120 and thereby select, capture, and/or move individual droplets or groups of droplets.

One example of a microfluidic device 200 comprising a DEP configuration is illustrated in FIGS. 2A and 2B. While for purposes of simplicity FIGS. 2A and 2B show a side cross-sectional view and a top cross-sectional view, respectively, of a portion of an enclosure 102 of the microfluidic device 200 having an open region/chamber 202, it should be understood that the region/chamber 202 may be part of a fluidic circuit element having a more detailed structure, such as a growth chamber, an isolation pen, a flow region, or a flow channel. Furthermore, the microfluidic device 200 may include other fluidic circuit elements. For example, the microfluidic device 200 can include a plurality of growth chambers or isolation pens and/or one or more flow regions or flow channels, such as those described herein with respect to microfluidic device 100. A DEP configuration may be incorporated into any such fluidic circuit elements of the microfluidic device 200, or select portions thereof. It should be further appreciated that any of the above or below described microfluidic device components and system components may be incorporated in and/or used in combination with the microfluidic device 200. For example, system 150 including control and monitoring equipment 152, described above, may be used with microfluidic device 200, including one or more of the media module 160, motive module 162, imaging module 164, tilting module 166, and other modules 168.

As seen in FIG. 2A, the microfluidic device 200 includes a support structure 104 having a bottom electrode 204 and an electrode activation substrate 206 overlying the bottom electrode 204, and a cover 110 having a top electrode 210, with the top electrode 210 spaced apart from the bottom electrode 204. The top electrode 210 and the electrode activation substrate 206 define opposing surfaces of the region/chamber 202. A medium 180 contained in the region/chamber 202 thus provides a resistive connection between the top electrode 210 and the electrode activation substrate 206. A power source 212 configured to be connected to the bottom electrode 204 and the top electrode 210 and create a biasing voltage between the electrodes, as required for the generation of DEP forces in the region/chamber 202, is also shown. The power source 212 can be, for example, an alternating current (AC) power source.

In certain embodiments, the microfluidic device 200 illustrated in FIGS. 2A and 2B can have an optically-actuated DEP configuration. Accordingly, changing patterns of light 222 from the light source 220, which may be controlled by the motive module 162, can selectively activate and deactivate changing patterns of DEP electrodes at regions 214 of the inner surface 208 of the electrode activation substrate 206. (Hereinafter the regions 214 of a microfluidic device having a DEP configuration are referred to as “DEP electrode regions.”) As illustrated in FIG. 2B, a light pattern 222 directed onto the inner surface 208 of the electrode activation substrate 206 can illuminate select DEP electrode regions 214a (shown in white) in a pattern, such as a square. The non-illuminated DEP electrode regions 214 (cross-hatched) are hereinafter referred to as “dark” DEP electrode regions 214. The relative electrical impedance through the DEP electrode activation substrate 206 (i.e., from the bottom electrode 204 up to the inner surface 208 of the electrode activation substrate 206 which interfaces with the medium 180 in the flow region 106) is greater than the relative electrical impedance through the medium 180 in the region/chamber 202 (i.e., from the inner surface 208 of the electrode activation substrate 206 to the top electrode 210 of the cover 110) at each dark DEP electrode region 214. An illuminated DEP electrode region 214a, however, exhibits a reduced relative impedance through the electrode activation substrate 206 that is less than the relative impedance through the medium 180 in the region/chamber 202 at each illuminated DEP electrode region 214a.

With the power source 212 activated, the foregoing DEP configuration creates an electric field gradient in the fluidic medium 180 between illuminated DEP electrode regions 214a and adjacent dark DEP electrode regions 214, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 214 at the inner surface 208 of the region/chamber 202 by changing light patterns 222 projected from a light source 220 into the microfluidic device 200. Whether the DEP forces attract or repel nearby micro-objects can depend on such parameters as the frequency of the power source 212 and the dielectric properties of the medium 180 and/or micro-objects (not shown).

The square pattern 224 of illuminated DEP electrode regions 214a illustrated in FIG. 2B is an example only. Any pattern of the DEP electrode regions 214 can be illuminated (and thereby activated) by the pattern of light 222 projected into the device 200, and the pattern of illuminated/activated DEP electrode regions 214 can be repeatedly changed by changing or moving the light pattern 222.

In some embodiments, the electrode activation substrate 206 can comprise or consist of a photoconductive material. In such embodiments, the inner surface 208 of the electrode activation substrate 206 can be featureless. For example, the electrode activation substrate 206 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 214 can be created anywhere and in any pattern on the inner surface 208 of the electrode activation substrate 208, in accordance with the light pattern 222. The number and pattern of the DEP electrode regions 214 thus need not be fixed, but can correspond to the light pattern 222. Examples of microfluidic devices having a DEP configuration comprising a photoconductive layer such as discussed above have been described, for example, in U.S. Pat. No. RE 44,711 (Wu et al.) (originally issued as U.S. Pat. No. 7,612,355), the entire contents of which are incorporated herein by reference.

In other embodiments, the electrode activation substrate 206 can comprise a substrate comprising a plurality of doped layers, electrically insulating layers (or regions), and electrically conductive layers that form semiconductor integrated circuits, such as is known in semiconductor fields. For example, the electrode activation substrate 206 can comprise a plurality of phototransistors, including, for example, lateral bipolar phototransistors, each phototransistor corresponding to a DEP electrode region 214. Alternatively, the electrode activation substrate 206 can comprise electrodes (e.g., conductive metal electrodes) controlled by phototransistor switches, with each such electrode corresponding to a DEP electrode region 214. The electrode activation substrate 206 can include a pattern of such phototransistors or phototransistor-controlled electrodes. The pattern, for example, can be an array of substantially square phototransistors or phototransistor-controlled electrodes arranged in rows and columns, such as shown in FIG. 2B. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 214 at the inner surface 208 of the electrode activation substrate 206 and the bottom electrode 210, and those electrical connections (i.e., phototransistors or electrodes) can be selectively activated and deactivated by the light pattern 222. When not activated, each electrical connection can have high impedance such that the relative impedance through the electrode activation substrate 206 (i.e., from the bottom electrode 204 to the inner surface 208 of the electrode activation substrate 206 which interfaces with the medium 180 in the region/chamber 202) is greater than the relative impedance through the medium 180 (i.e., from the inner surface 208 of the electrode activation substrate 206 to the top electrode 210 of the cover 110) at the corresponding DEP electrode region 214. When activated by light in the light pattern 222, however, the relative impedance through the electrode activation substrate 206 is less than the relative impedance through the medium 180 at each illuminated DEP electrode region 214, thereby activating the DEP electrode at the corresponding DEP electrode region 214 as discussed above. DEP electrodes that attract or repel micro-objects (not shown) in the medium 180 can thus be selectively activated and deactivated at many different DEP electrode regions 214 at the inner surface 208 of the electrode activation substrate 206 in the region/chamber 202 in a manner determined by the light pattern 222.

Examples of microfluidic devices having electrode activation substrates that comprise phototransistors have been described, for example, in U.S. Pat. No. 7,956,339 (Ohta et al.) (see, e.g., device 300 illustrated in FIGS. 21 and 22, and descriptions thereof), the entire contents of which are incorporated herein by reference. Examples of microfluidic devices having electrode activation substrates that comprise electrodes controlled by phototransistor switches have been described, for example, in U.S. Patent Publication No. 2014/0124370 (Short et al.) (see, e.g., devices 200, 400, 500, 600, and 900 illustrated throughout the drawings, and descriptions thereof), the entire contents of which are incorporated herein by reference.

In some embodiments of a DEP configured microfluidic device, the top electrode 210 is part of a first wall (or cover 110) of the enclosure 102, and the electrode activation substrate 206 and bottom electrode 204 are part of a second wall (or support structure 104) of the enclosure 102. The region/chamber 202 can be between the first wall and the second wall. In other embodiments, the electrode 210 is part of the second wall (or support structure 104) and one or both of the electrode activation substrate 206 and/or the electrode 210 are part of the first wall (or cover 110). Moreover, the light source 220 can alternatively be used to illuminate the enclosure 102 from below.

With the microfluidic device 200 of FIGS. 2A-2B having a DEP configuration, the motive module 162 can select a micro-object (not shown) in the medium 180 in the region/chamber 202 by projecting a light pattern 222 into the device 200 to activate a first set of one or more DEP electrodes at DEP electrode regions 214a of the inner surface 208 of the electrode activation substrate 206 in a pattern (e.g., square pattern 224) that surrounds and captures the micro-object. The motive module 162 can then move the captured micro-object by moving the light pattern 222 relative to the device 200 to activate a second set of one or more DEP electrodes at DEP electrode regions 214. Alternatively, the device 200 can be moved relative to the light pattern 222.

In other embodiments, the microfluidic device 200 can have a DEP configuration that does not rely upon light activation of DEP electrodes at the inner surface 208 of the electrode activation substrate 206. For example, the electrode activation substrate 206 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 214, thereby creating a net DEP force on a micro-object (not shown) in region/chamber 202 in the vicinity of the activated DEP electrodes. Depending on such characteristics as the frequency of the power source 212 and the dielectric properties of the medium (not shown) and/or micro-objects in the region/chamber 202, the DEP force can attract or repel a nearby micro-object. By selectively activating and deactivating a set of DEP electrodes (e.g., at a set of DEP electrodes regions 214 that forms a square pattern 224), one or more micro-objects in region/chamber 202 can be trapped and moved within the region/chamber 202. The motive module 162 in FIG. 1 can control such switches and thus activate and deactivate individual ones of the DEP electrodes to select, trap, and move particular micro-objects (not shown) around the region/chamber 202. Microfluidic devices having a DEP configuration that includes selectively addressable and energizable electrodes are known in the art and have been described, for example, in U.S. Pat. No. 6,294,063 (Becker et al.) and U.S. Pat. No. 6,942,776 (Medoro), the entire contents of which are incorporated herein by reference.

As yet another example, the microfluidic device 200 can have an electrowetting (EW) configuration, which can be in place of the DEP configuration or can be located in a portion of the microfluidic device 200 that is separate from the portion which has the DEP configuration. The EW configuration can be an opto-electrowetting configuration or an electrowetting on dielectric (EWOD) configuration, both of which are known in the art. In some EW configurations, the support structure 104 has an electrode activation substrate 206 sandwiched between a dielectric layer (not shown) and the bottom electrode 204. The dielectric layer can comprise a hydrophobic material and/or can be coated with a hydrophobic material. For microfluidic devices 200 that have an EW configuration, the inner surface 208 of the support structure 104 is the inner surface of the dielectric layer or its hydrophobic coating.

The dielectric layer (not shown) can comprise one or more oxide layers, and can have a thickness of about 50 nm to about 250 nm (e.g., about 125 nm to about 175 nm). In certain embodiments, the dielectric layer may comprise a layer of oxide, such as a metal oxide (e.g., aluminum oxide or halfnuim oxide). In certain embodiments, the dielectric layer can comprise a dielectric material other than a metal oxide, such as silicon oxide or a nitride. Regardless of the exact composition and thickness, the dielectric layer can have an impedance of about 10 kOhms to about 50 kOhms.

In some embodiments, the surface of the dielectric layer that faces inward toward region/chamber 202 is coated with a hydrophobic material. The hydrophobic material can comprise, for example, fluorinated carbon molecules. Examples of fluorinated carbon molecules include perfluoro-polymers such as polytetrafluoroethylene (e.g., TEFLON®) or poly(2,3-difluoromethylenyl-perfluorotetrahydrofuran) (e.g., CYTOP™). Molecules that make up the hydrophobic material can be covalently bonded to the surface of the dielectric layer. For example, molecules of the hydrophobic material can be covalently bound to the surface of the dielectric layer by means of a linker such as a siloxane group, a phosphonic acid group, or a thiol group. Thus, in some embodiments, the hydrophobic material can comprise alkyl-terminated siloxane, alkyl-termination phosphonic acid, or alkyl-terminated thiol. The alkyl group can be long-chain hydrocarbons (e.g., having a chain of at least 10 carbons, or at least 16, 18, 20, 22, or more carbons). Alternatively, fluorinated (or perfluorinated) carbon chains can be used in place of the alkyl groups. Thus, for example, the hydrophobic material can comprise fluoroalkyl-terminated siloxane, fluoroalkyl-terminated phosphonic acid, or fluoroalkyl-terminated thiol. In some embodiments, the hydrophobic coating has a thickness of about 10 nm to about 50 nm. In other embodiments, the hydrophobic coating has a thickness of less than 10 nm (e.g., less than 5 nm, or about 1.5 to 3.0 nm).

In some embodiments, the cover 110 of a microfluidic device 200 having an electrowetting configuration is coated with a hydrophobic material (not shown) as well. The hydrophobic material can be the same hydrophobic material used to coat the dielectric layer of the support structure 104, and the hydrophobic coating can have a thickness that is substantially the same as the thickness of the hydrophobic coating on the dielectric layer of the support structure 104. Moreover, the cover 110 can comprise an electrode activation substrate 206 sandwiched between a dielectric layer and the top electrode 210, in the manner of the support structure 104. The electrode activation substrate 206 and the dielectric layer of the cover 110 can have the same composition and/or dimensions as the electrode activation substrate 206 and the dielectric layer of the support structure 104. Thus, the microfluidic device 200 can have two electrowetting surfaces.

In some embodiments, the electrode activation substrate 206 can comprise a photoconductive material, such as described above. Accordingly, in certain embodiments, the electrode activation substrate 206 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. Alternatively, the electrode activation substrate 206 can comprise electrodes (e.g., conductive metal electrodes) controlled by phototransistor switches, as described above. Microfluidic devices having an opto-electrowetting configuration are known in the art and/or can be constructed with electrode activation substrates known in the art. For example, U.S. Pat. No. 6,958,132 (Chiou et al.), the entire contents of which are incorporated herein by reference, discloses opto-electrowetting configurations having a photoconductive material such as a-Si:H, while U.S. Patent Publication No. 2014/0124370 (Short et al.), referenced above, discloses electrode activation substrates having electrodes controlled by phototransistor switches.

The microfluidic device 200 thus can have an opto-electrowetting configuration, and light patterns 222 can be used to activate photoconductive EW regions or photoresponsive EW electrodes in the electrode activation substrate 206. Such activated EW regions or EW electrodes of the electrode activation substrate 206 can generate an electrowetting force at the inner surface 208 of the support structure 104 (i.e., the inner surface of the overlaying dielectric layer or its hydrophobic coating). By changing the light patterns 222 (or moving microfluidic device 200 relative to the light source 220) incident on the electrode activation substrate 206, droplets (e.g., containing an aqueous medium, solution, or solvent) contacting the inner surface 208 of the support structure 104 can be moved through an immiscible fluid (e.g., an oil medium) present in the region/chamber 202.

In other embodiments, microfluidic devices 200 can have an EWOD configuration, and the electrode activation substrate 206 can comprise selectively addressable and energizable electrodes that do not rely upon light for activation. The electrode activation substrate 206 thus can include a pattern of such electrowetting (EW) electrodes. The pattern, for example, can be an array of substantially square EW electrodes arranged in rows and columns, such as shown in FIG. 2B. Alternatively, the pattern can be an array of substantially hexagonal EW electrodes that form a hexagonal lattice. Regardless of the pattern, the EW electrodes can be selectively activated (or deactivated) by electrical switches (e.g., transistor switches in a semiconductor substrate). By selectively activating and deactivating EW electrodes in the electrode activation substrate 206, droplets (not shown) contacting the inner surface 208 of the overlaying dielectric layer or its hydrophobic coating can be moved within the region/chamber 202. The motive module 162 in FIG. 1 can control such switches and thus activate and deactivate individual EW electrodes to select and move particular droplets around region/chamber 202. Microfluidic devices having a EWOD configuration with selectively addressable and energizable electrodes are known in the art and have been described, for example, in U.S. Pat. No. 8,685,344 (Sundarsan et al.), the entire contents of which are incorporated herein by reference.

Regardless of the configuration of the microfluidic device 200, a power source 212 can be used to provide a potential (e.g., an AC voltage potential) that powers the electrical circuits of the microfluidic device 200. The power source 212 can be the same as, or a component of, the power source 192 referenced in FIG. 1. Power source 212 can be configured to provide an AC voltage and/or current to the top electrode 210 and the bottom electrode 204. For an AC voltage, the power source 212 can provide a frequency range and an average or peak power (e.g., voltage or current) range sufficient to generate net DEP forces (or electrowetting forces) strong enough to trap and move individual micro-objects (not shown) in the region/chamber 202, as discussed above, and/or to change the wetting properties of the inner surface 208 of the support structure 104 (i.e., the dielectric layer and/or the hydrophobic coating on the dielectric layer) in the region/chamber 202, as also discussed above. Such frequency ranges and average or peak power ranges are known in the art. See, e.g., U.S. Pat. No. 6,958,132 (Chiou et al.), U.S. Pat. No. RE44,711 (Wu et al.) (originally issued as U.S. Pat. No. 7,612,355), and US Patent Application Publication Nos. US2014/0124370 (Short et al.), US2015/0306598 (Khandros et al.), and US2015/0306599 (Khandros et al.).

Isolation Pens. Non-limiting examples of generic isolation pens 244, 246, and 248 are shown within the microfluidic device 240 depicted in FIGS. 2C and 2D. Each isolation pen 244, 246, and 248 can comprise an isolation structure 250 defining an isolation region 258 and a connection region 254 fluidically connecting the isolation region 258 to a channel 122. The connection region 254 can comprise a proximal opening 252 to the channel 122 and a distal opening 256 to the isolation region 258. The connection region 254 can be configured so that the maximum penetration depth of a flow of a fluidic medium (not shown) flowing from the channel 122 into the isolation pen 244, 246, 248 does not extend into the isolation region 258. Thus, due to the connection region 254, a micro-object (not shown) or other material (not shown) disposed in an isolation region 258 of an isolation pen 244, 246, 248 can thus be isolated from, and not substantially affected by, a flow of medium 180 in the channel 122.

The channel 122 can thus be an example of a swept region, and the isolation regions 258 of the isolation pens 244, 246, 248 can be examples of unswept regions. As noted, the channel 122 and isolation pens 244, 246, 248 can be configured to contain one or more fluidic media 180. In the example shown in FIGS. 2C-2D, the ports 242 are connected to the channel 122 and allow a fluidic medium 180 to be introduced into or removed from the microfluidic device 240. Prior to introduction of the fluidic medium 180, the microfluidic device may be primed with a gas such as carbon dioxide gas. Once the microfluidic device 240 contains the fluidic medium 180, the flow 260 of fluidic medium 180 in the channel 122 can be selectively generated and stopped. For example, as shown, the ports 242 can be disposed at different locations (e.g., opposite ends) of the channel 122, and a flow 260 of medium can be created from one port 242 functioning as an inlet to another port 242 functioning as an outlet.

FIG. 2E illustrates a detailed view of an example of an isolation pen 244 according to the present invention. Examples of micro-objects 270 are also shown.

As is known, a flow 260 of fluidic medium 180 in a microfluidic channel 122 past a proximal opening 252 of isolation pen 244 can cause a secondary flow 262 of the medium 180 into and/or out of the isolation pen 244. To isolate micro-objects 270 in the isolation region 258 of an isolation pen 244 from the secondary flow 262, the length L_{con} of the connection region 254 of the isolation pen 244 (i.e., from the proximal opening 252 to the distal opening 256) should be greater than the penetration depth Dₚ of the secondary flow 262 into the connection region 254. The penetration depth Dₚ of the secondary flow 262 depends upon the velocity of the fluidic medium 180 flowing in the channel 122 and various parameters relating to the configuration of the channel 122 and the proximal opening 252 of the connection region 254 to the channel 122. For a given microfluidic device, the configurations of the channel 122 and the opening 252 will be fixed, whereas the rate of flow 260 of fluidic medium 180 in the channel 122 will be variable. Accordingly, for each isolation pen 244, a maximal velocity Vₘₐₓ for the flow 260 of fluidic medium 180 in channel 122 can be identified that ensures that the penetration depth Dₚ of the secondary flow 262 does not exceed the length L_{con} of the connection region 254. As long as the rate of the flow 260 of fluidic medium 180 in the channel 122 does not exceed the maximum velocity Vₘₐₓ, the resulting secondary flow 262 can be limited to the channel 122 and the connection region 254 and kept out of the isolation region 258. The flow 260 of medium 180 in the channel 122 will thus not draw micro-objects 270 out of the isolation region 258. Rather, micro-objects 270 located in the isolation region 258 will stay in the isolation region 258 regardless of the flow 260 of fluidic medium 180 in the channel 122.

Moreover, as long as the rate of flow 260 of medium 180 in the channel 122 does not exceed Vₘₐₓ, the flow 260 of fluidic medium 180 in the channel 122 will not move miscellaneous particles (e.g., microparticles and/or nanoparticles) from the channel 122 into the isolation region 258 of an isolation pen 244. Having the length L_{con} of the connection region 254 be greater than the maximum penetration depth Dₚ of the secondary flow 262 can thus prevent contamination of one isolation pen 244 with miscellaneous particles from the channel 122 or another isolation pen (e.g., isolation pens 246, 248 in FIG. 2D).

Because the channel 122 and the connection regions 254 of the isolation pens 244, 246, 248 can be affected by the flow 260 of medium 180 in the channel 122, the channel 122 and connection regions 254 can be deemed swept (or flow) regions of the microfluidic device 240. The isolation regions 258 of the isolation pens 244, 246, 248, on the other hand, can be deemed unswept (or non-flow) regions. For example, components (not shown) in a first fluidic medium 180 in the channel 122 can mix with a second fluidic medium 280 in the isolation region 258 substantially only by diffusion of components of the first medium 180 from the channel 122 through the connection region 254 and into the second fluidic medium 280 in the isolation region 258. Similarly, components (not shown) of the second medium 280 in the isolation region 258 can mix with the first medium 180 in the channel 122 substantially only by diffusion of components of the second medium 280 from the isolation region 258 through the connection region 254 and into the first medium 180 in the channel 122. The first medium 180 can be the same medium or a different medium than the second medium 280. Moreover, the first medium 180 and the second medium 280 can start out being the same, then become different (e.g., through conditioning of the second medium 280 by one or more cells in the isolation region 258, or by changing the medium 180 flowing through the channel 122).

The maximum penetration depth Dₚ of the secondary flow 262 caused by the flow 260 of fluidic medium 180 in the channel 122 can depend on a number of parameters, as mentioned above. Examples of such parameters include: the shape of the channel 122 (e.g., the channel can direct medium into the connection region 254, divert medium away from the connection region 254, or direct medium in a direction substantially perpendicular to the proximal opening 252 of the connection region 254 to the channel 122); a width W_{ch} (or cross-sectional area) of the channel 122 at the proximal opening 252; and a width W_{con} (or cross-sectional area) of the connection region 254 at the proximal opening 252; the velocity V of the flow 260 of fluidic medium 180 in the channel 122; the viscosity of the first medium 180 and/or the second medium 280, or the like.

In some embodiments, the dimensions of the channel 122 and isolation pens 244, 246, 248 can be oriented as follows with respect to the vector of the flow 260 of fluidic medium 180 in the channel 122: the channel width W_{ch} (or cross-sectional area of the channel 122) can be substantially perpendicular to the flow 260 of medium 180; the width W_{con} (or cross-sectional area) of the connection region 254 at opening 252 can be substantially parallel to the flow 260 of medium 180 in the channel 122; and/or the length L_{con} of the connection region can be substantially perpendicular to the flow 260 of medium 180 in the channel 122. The foregoing are examples only, and the relative position of the channel 122 and isolation pens 244, 246, 248 can be in other orientations with respect to each other.

As illustrated in FIG. 2E, the width W_{con} of the connection region 254 can be uniform from the proximal opening 252 to the distal opening 256. The width W_{con} of the connection region 254 at the distal opening 256 can thus be in any of the ranges identified herein for the width W_{con} of the connection region 254 at the proximal opening 252. Alternatively, the width W_{con} of the connection region 254 at the distal opening 256 can be larger than the width W_{con} of the connection region 254 at the proximal opening 252.

As illustrated in FIG. 2E, the width of the isolation region 258 at the distal opening 256 can be substantially the same as the width W_{con} of the connection region 254 at the proximal opening 252. The width of the isolation region 258 at the distal opening 256 can thus be in any of the ranges identified herein for the width W_{con} of the connection region 254 at the proximal opening 252. Alternatively, the width of the isolation region 258 at the distal opening 256 can be larger or smaller than the width W_{con} of the connection region 254 at the proximal opening 252. Moreover, the distal opening 256 may be smaller than the proximal opening 252 and the width W_{con} of the connection region 254 may be narrowed between the proximal opening 252 and distal opening 256. For example, the connection region 254 may be narrowed between the proximal opening and the distal opening, using a variety of different geometries (e.g. chamfering the connection region, beveling the connection region). Further, any part or subpart of the connection region 254 may be narrowed (e.g. a portion of the connection region adjacent to the proximal opening 252).

In various embodiments of isolation pens (e.g. 124, 126, 128, 130, 244, 246 or 248), the isolation region (e.g. 258) is configured to contain a plurality of micro-objects. In other embodiments, the isolation region can be configured to contain only one, two, three, four, five, or a similar relatively small number of micro-objects. Accordingly, the volume of an isolation region can be, for example, at least 4×10⁵, 8×10⁵, 1×10⁶, 2×10⁶, 4×10⁶, 6×10⁶ cubic microns, or more.

In various embodiments of isolation pens, the width W_{ch} of the channel 122 at a proximal opening (e.g. 252) can be within any of the following ranges: 50-1000 microns, 50-500 microns, 50-400 microns, 50-300 microns, 50-250 microns, 50-200 microns, 50-150 microns, 50-100 microns, 70-500 microns, 70-400 microns, 70-300 microns, 70-250 microns, 70-200 microns, 70-150 microns, 90-400 microns, 90-300 microns, 90-250 microns, 90-200 microns, 90-150 microns, 100-300 microns, 100-250 microns, 100-200 microns, 100-150 microns, and 100-120 microns. The foregoing are examples only, and the width W_{ch} of the channel 122 can be in other ranges (e.g., a range defined by any of the endpoints listed above). Moreover, the W_{ch} of the channel 122 can be selected to be in any of these ranges in regions of the channel other than at a proximal opening of an isolation pen.

In some embodiments, an isolation pen has a cross-sectional height of about 30 to about 200 microns, or about 50 to about 150 microns. In some embodiments, the isolation pen has a cross-sectional area of about 100,000 to about 2,500,000 square microns, or about 200,000 to about 2,000,000 square microns. In some embodiments, a connection region has a cross-sectional height that matches the cross-sectional height of the corresponding isolation pen. In some embodiments, the connection region has a cross-sectional width of about 50 to about 500 microns, or about 100 to about 300 microns.

In various embodiments of isolation pens, the height H_{ch} of the channel 122 at a proximal opening 252 can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{ch} of the channel 122 can be in other ranges (e.g., a range defined by any of the endpoints listed above). The height H_{ch} of the channel 122 can be selected to be in any of these ranges in regions of the channel other than at a proximal opening of an isolation pen.

In various embodiments of isolation pens a cross-sectional area of the channel 122 at a proximal opening 252 can be within any of the following ranges: 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the channel 122 at a proximal opening 252 can be in other ranges (e.g., a range defined by any of the endpoints listed above).

In various embodiments of isolation pens, the length L_{con} of the connection region 254 can be in any of the following ranges: 1-200 microns, 5-150 microns, 10-100 microns, 15-80 microns, 20-60 microns, 20-500 microns, 40-400 microns, 60-300 microns, 80-200 microns, and 100-150 microns. The foregoing are examples only, and length L_{con} of a connection region 254 can be in a different range than the foregoing examples (e.g., a range defined by any of the endpoints listed above).

In various embodiments of isolation pens the width W_{con} of a connection region 254 at a proximal opening 252 can be in any of the following ranges: 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-250 microns, 50-200 microns, 50-150 microns, 50-100 microns, 50-80 microns, 60-200 microns, 60-150 microns, 60-100 microns, 60-80 microns, 70-150 microns, 70-100 microns, and 80-100 microns. The foregoing are examples only, and the width W_{con} of a connection region 254 at a proximal opening 252 can be different than the foregoing examples (e.g., a range defined by any of the endpoints listed above).

In various embodiments of isolation pens, the width W_{con} of a connection region 254 at a proximal opening 252 can be at least as large as the largest dimension of a micro-object (e.g., oocyte, ovum, embryo, sperm) that the isolation pen is intended for. For example, the width W_{con} of a connection region 254 at a proximal opening 252 of an isolation pen that an oocyte, ovum, or embryo will be placed into can be in any of the following ranges: about 100 microns, about 110 microns, about 120 microns, about 130 microns, about 140 microns, about 150 microns, about 160 microns, about 170 microns, about 180 microns, about 190 microns, about 200 microns, or about 100-200 microns, about 120-200 microns, or about 140-200 microns. The foregoing are examples only, and the width W_{con} of a connection region 254 at a proximal opening 252 can be different than the foregoing examples (e.g., a range defined by any of the endpoints listed above).

In various embodiments of isolation pens, a ratio of the length L_{con} of a connection region 254 to a width W_{con} of the connection region 254 at the proximal opening 252 can be greater than or equal to any of the following ratios: 0.5, 1.0, 1.5, 2.0, 2.5, 3.0, 3.5, 4.0, 4.5, 5.0, 6.0, 7.0, 8.0, 9.0, 10.0, or more. The foregoing are examples only, and the ratio of the length L_{con} of a connection region 254 to a width W_{con} of the connection region 254 at the proximal opening 252 can be different than the foregoing examples.

In various embodiments of microfluidic devices 100, 200, 240, 290, Vₘₐₓ can be set around 0.2, 0.3, 0.4, 0.5, 0.6, 0.7, 0.8, 0.9, 1.0, 1.1, 1.2, 1.3, 1.4, or 1.5 μL/sec.

In various embodiments of microfluidic devices having isolation pens, the volume of an isolation region 258 of an isolation pen can be, for example, at least 5×10⁵, 8×10⁵, 1×10⁶, 2×10⁶, 4×10⁶, 6×10⁶, 8×10⁶, 1×10⁷ cubic microns, or more. In various embodiments of microfluidic devices having isolation pens, the volume of an isolation pen may be about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more.

In various embodiment, the microfluidic device has isolation pens configured as in any of the embodiments discussed herein where the microfluidic device has about 5 to about 10 isolation pens, about 10 to about 50 isolation pens, about 100 to about 500 isolation pens; about 200 to about 1000 isolation pens, or about 500 to about 1500 isolation pens. Such isolation pens need not all be the same size.

FIG. 2F illustrates a microfluidic device 290 according to one embodiment. The microfluidic device 290 is illustrated in FIG. 2F is a stylized diagram of a microfluidic device 100. In practice the microfluidic device 290 and its constituent circuit elements (e.g. channels 122 and isolation pens 128) would have the dimensions discussed herein. The microfluidic circuit 120 illustrated in FIG. 2F has two ports 107, four distinct channels 122 and four distinct flow paths 106. The microfluidic device 290 further comprises a plurality of isolation pens opening off of each channel 122. In the microfluidic device illustrated in FIG. 2F, the isolation pens have a geometry similar to the pens illustrated in FIG. 2E and thus, have both connection regions and isolation regions. Accordingly, the microfluidic circuit 120 includes both swept regions (e.g. channels 122 and portions of the connection regions 254 within the maximum penetration depth Dₚ of the secondary flow 262) and non-swept regions (e.g. isolation regions 258 and portions of the connection regions 254 not within the maximum penetration depth Dₚ of the secondary flow 262).

FIGS. 3A through 3B shows various embodiments of system 150 which can be used to operate and observe microfluidic devices (e.g. 100, 200, 240, 290) according to the present invention. As illustrated in FIG. 3A, the system 150 can include a structure (“nest”) 300 configured to hold a microfluidic device 100 (not shown), or any other microfluidic device described herein. The nest 300 can include a socket 302 capable of interfacing with the microfluidic device 360 (e.g., an optically-actuated electrokinetic device 100) and providing electrical connections from power source 192 to microfluidic device 360. The nest 300 can further include an integrated electrical signal generation subsystem 304. The electrical signal generation subsystem 304 can be configured to supply a biasing voltage to socket 302 such that the biasing voltage is applied across a pair of electrodes in the microfluidic device 360 when it is being held by socket 302. Thus, the electrical signal generation subsystem 304 can be part of power source 192. The ability to apply a biasing voltage to microfluidic device 360 does not mean that a biasing voltage will be applied at all times when the microfluidic device 360 is held by the socket 302. Rather, in most cases, the biasing voltage will be applied intermittently, e.g., only as needed to facilitate the generation of electrokinetic forces, such as dielectrophoresis or electro-wetting, in the microfluidic device 360.

As illustrated in FIG. 3A, the nest 300 can include a printed circuit board assembly (PCBA) 320. The electrical signal generation subsystem 304 can be mounted on and electrically integrated into the PCBA 320. The exemplary support includes socket 302 mounted on PCBA 320, as well.

Typically, the electrical signal generation subsystem 304 will include a waveform generator (not shown). The electrical signal generation subsystem 304 can further include an oscilloscope (not shown) and/or a waveform amplification circuit (not shown) configured to amplify a waveform received from the waveform generator. The oscilloscope, if present, can be configured to measure the waveform supplied to the microfluidic device 360 held by the socket 302. In certain embodiments, the oscilloscope measures the waveform at a location proximal to the microfluidic device 360 (and distal to the waveform generator), thus ensuring greater accuracy in measuring the waveform actually applied to the device. Data obtained from the oscilloscope measurement can be, for example, provided as feedback to the waveform generator, and the waveform generator can be configured to adjust its output based on such feedback. An example of a suitable combined waveform generator and oscilloscope is the Red Pitaya™.

In certain embodiments, the nest 300 further comprises a controller 308, such as a microprocessor used to sense and/or control the electrical signal generation subsystem 304. Examples of suitable microprocessors include the Arduino™ microprocessors, such as the Arduino Nano™. The controller 308 may be used to perform functions and analysis or may communicate with an external master controller 154 (shown in FIG. 1) to perform functions and analysis. In the embodiment illustrated in FIG. 3A the controller 308 communicates with a master controller 154 through an interface 310 (e.g., a plug or connector).

In some embodiments, the nest 300 can comprise an electrical signal generation subsystem 304 comprising a Red Pitaya™ waveform generator/oscilloscope unit (“Red Pitaya unit”) and a waveform amplification circuit that amplifies the waveform generated by the Red Pitaya unit and passes the amplified voltage to the microfluidic device 100. In some embodiments, the Red Pitaya unit is configured to measure the amplified voltage at the microfluidic device 360 and then adjust its own output voltage as needed such that the measured voltage at the microfluidic device 360 is the desired value. In some embodiments, the waveform amplification circuit can have a +6.5V to −6.5V power supply generated by a pair of DC-DC converters mounted on the PCBA 320, resulting in a signal of up to 13 Vpp at the microfluidic device 100.

As illustrated in FIG. 3A, the support structure 300 can further include a thermal control subsystem 306. The thermal control subsystem 306 can be configured to regulate the temperature of microfluidic device 360 held by the support structure 300. For example, the thermal control subsystem 306 can include a Peltier thermoelectric device (not shown) and a cooling unit (not shown). The Peltier thermoelectric device can have a first surface configured to interface with at least one surface of the microfluidic device 360. The cooling unit can be, for example, a cooling block (not shown), such as a liquid-cooled aluminum block. A second surface of the Peltier thermoelectric device (e.g., a surface opposite the first surface) can be configured to interface with a surface of such a cooling block. The cooling block can be connected to a fluidic path 330 configured to circulate cooled fluid through the cooling block. In the embodiment illustrated in FIG. 3A, the support structure 300 comprises an inlet 332 and an outlet 334 to receive cooled fluid from an external reservoir (not shown), introduce the cooled fluid into the fluidic path 330 and through the cooling block, and then return the cooled fluid to the external reservoir. In some embodiments, the Peltier thermoelectric device, the cooling unit, and/or the fluidic path 330 can be mounted on a casing 340 of the support structure 300. In some embodiments, the thermal control subsystem 306 is configured to regulate the temperature of the Peltier thermoelectric device so as to achieve a target temperature for the microfluidic device 360. Temperature regulation of the Peltier thermoelectric device can be achieved, for example, by a thermoelectric power supply, such as a Pololu™ thermoelectric power supply (Pololu Robotics and Electronics Corp.). The thermal control subsystem 306 can include a feedback circuit, such as a temperature value provided by an analog circuit. Alternatively, the feedback circuit can be provided by a digital circuit.

In some embodiments, the nest 300 can include a thermal control subsystem 306 with a feedback circuit that is an analog voltage divider circuit (not shown) which includes a resistor (e.g., with resistance 1 kOhm+/−0.1%, temperature coefficient+/−0.02 ppm/CO) and a NTC thermistor (e.g., with nominal resistance 1 kOhm+/−0.01%). In some instances, the thermal control subsystem 306 measures the voltage from the feedback circuit and then uses the calculated temperature value as input to an on-board PID control loop algorithm. Output from the PID control loop algorithm can drive, for example, both a directional and a pulse-width-modulated signal pin on a Pololu™ motor drive (not shown) to actuate the thermoelectric power supply, thereby controlling the Peltier thermoelectric device.

The nest 300 can include a serial port 350 which allows the microprocessor of the controller 308 to communicate with an external master controller 154 via the interface 310. In addition, the microprocessor of the controller 308 can communicate (e.g., via a Plink tool (not shown)) with the electrical signal generation subsystem 304 and thermal control subsystem 306. Thus, via the combination of the controller 308, the interface 310, and the serial port 350, the electrical signal generation subsystem 308 and the thermal control subsystem 306 can communicate with the external master controller 154. In this manner, the master controller 154 can, among other things, assist the electrical signal generation subsystem 308 by performing scaling calculations for output voltage adjustments. A Graphical User Interface (GUI) (not shown) provided via a display device 170 coupled to the external master controller 154, can be configured to plot temperature and waveform data obtained from the thermal control subsystem 306 and the electrical signal generation subsystem 308, respectively. Alternatively, or in addition, the GUI can allow for updates to the controller 308, the thermal control subsystem 306, and the electrical signal generation subsystem 304.

As discussed above, system 150 can include an imaging device 194. In some embodiments, the imaging device 194 comprises a light modulating subsystem 404. The light modulating subsystem 404 can include a digital mirror device (DMD), or a microshutter array system (MSA), either of which can be configured to receive light from a light source 402 and transmits a subset of the received light into an optical train of microscope 400. Alternatively, the light modulating subsystem 404 can include a device that produces its own light (and thus dispenses with the need for a light source 402), such as an organic light emitting diode display (OLED), a liquid crystal on silicon (LCOS) device, a ferroelectric liquid crystal on silicon device (FLCOS), or a transmissive liquid crystal display (LCD). The light modulating subsystem 404 can be, for example, a projector. Thus, the light modulating subsystem 404 can be capable of emitting both structured and unstructured light. One example of a suitable light modulating subsystem 404 is the Mosaic™ system from Andor Technologies™. In certain embodiments, imaging module 164 and/or motive module 162 of system 150 can control the light modulating subsystem 404.

In certain embodiments, the imaging device 194 further comprises a microscope 400. In such embodiments, the nest 300 and light modulating subsystem 404 can be individually configured to be mounted on the microscope 400. The microscope 400 can be, for example, a standard research-grade light microscope or fluorescence microscope. Thus, the nest 300 can be configured to be mounted on the stage 410 of the microscope 400 and/or the light modulating subsystem 404 can be configured to mount on a port of microscope 400. In other embodiments, the nest 300 and the light modulating subsystem 404 described herein can be integral components of microscope 400.

In certain embodiments, the microscope 400 can further include one or more detectors 422. In some embodiments, the detector 422 is controlled by the imaging module 164. The detector 422 can include an eye piece, a charge-coupled device (CCD), a camera (e.g., a digital camera), or any combination thereof. If at least two detectors 422 are present, one detector can be, for example, a fast-frame-rate camera while the other detector can be a high sensitivity camera. Furthermore, the microscope 400 can include an optical train configured to receive reflected and/or emitted light from the microfluidic device 360 and focus at least a portion of the reflected and/or emitted light on the one or more detectors 422. The optical train of the microscope can also include different tube lenses (not shown) for the different detectors, such that the final magnification on each detector can be different.

In certain embodiments, imaging device 194 is configured to use at least two light sources. For example, a first light source 402 can be used to produce structured light (e.g., via the light modulating subsystem 404) and a second light source 432 can be used to provide unstructured light. The first light source 402 can produce structured light for optically-actuated electrokinesis and/or fluorescent excitation, and the second light source 432 can be used to provide bright field illumination. In these embodiments, the motive module 164 can be used to control the first light source 404 and the imaging module 164 can be used to control the second light source 432. The optical train of the microscope 400 can be configured to (1) receive structured light from the light modulating subsystem 404 and focus the structured light on at least a first region in a microfluidic device, such as an optically-actuated electrokinetic device, when the device is being held by the support structure 200, and (2) receive reflected and/or emitted light from the microfluidic device and focus at least a portion of such reflected and/or emitted light onto detector 422. The optical train can be further configured to receive unstructured light from a second light source and focus the unstructured light on at least a second region of the microfluidic device, when the device is held by the support structure 300. In certain embodiments, the first and second regions of the microfluidic device can be overlapping regions. For example, the first region can be a subset of the second region.

In FIG. 3B, the first light source 402 is shown supplying light to a light modulating subsystem 404, which provides structured light to the optical train of the microscope 400. The second light source 432 is shown providing unstructured light to the optical train via a beam splitter 436. Structured light from the light modulating subsystem 404 and unstructured light from the second light source 432 travel from the beam splitter 436 through the optical train together to reach a second beam splitter 436 (or dichroic filter 406, depending on the light provided by the light modulating subsystem 404), where the light gets reflected down through the objective 408 to the sample plane 412. Reflected and/or emitted light from the sample plane 412 then travels back up through the objective 408, through the beam splitter and/or dichroic filter 406, and to a dichroic filter 424. Only a fraction of the light reaching dichroic filter 424 passes through and reaches the detector 422.

In some embodiments, the second light source 432 emits blue light. With an appropriate dichroic filter 424, blue light reflected from the sample plane 412 is able to pass through dichroic filter 424 and reach the detector 422. In contrast, structured light coming from the light modulating subsystem 404 gets reflected from the sample plane 412, but does not pass through the dichroic filter 424. In this example, the dichroic filter 424 is filtering out visible light having a wavelength longer than 495 nm. Such filtering out of the light from the light modulating subsystem 404 would only be complete (as shown) if the light emitted from the light modulating subsystem did not include any wavelengths shorter than 495 nm. In practice, if the light coming from the light modulating subsystem 404 includes wavelengths shorter than 495 nm (e.g., blue wavelengths), then some of the light from the light modulating subsystem would pass through filter 424 to reach the detector 422. In such an embodiment, the filter 424 acts to change the balance between the amount of light that reaches the detector 422 from the first light source 402 and the second light source 432. This can be beneficial if the first light source 402 is significantly stronger than the second light source 432. In other embodiments, the second light source 432 can emit red light, and the dichroic filter 424 can filter out visible light other than red light (e.g., visible light having a wavelength shorter than 650 nm).

### EXAMPLES

### Example 1

### Monitoring an Embryo for High Viability

As shown in FIG. 4, an embryo can be placed in an isolation region 458 of an isolation pen 430 of a microfluidic device which may be configured as in any of microfluidic devices 100, 200, 240, or 290. The microfluidic device can have a flow path, which in this example is a channel 122 where the direction of flow 260 is indicated to be from left to right. The isolation pen is fluidically connected to the flow path, with the proximal opening 452 of the isolation pen opening to the channel such that the channel provides fresh medium all around the isolation pen (and embryo) without the fresh medium flowing into the isolation pen and carrying the embryo away. The channel walls 414 provide the boundaries of the flow path, and inner surface 208 of the microfluidic device may be the top surface of or overlay a substrate that may be configured as either a DEP configuration (including an optoelectronic tweezers (OET) configuration) or an electrowetting configuration (including an optoelectowetting (OEW) configuration). The placement of the embryo into the isolation pen may be performed by fluid flow, gravity, DEP, or electrowetting forces, or a combination thereof. The isolation region may be configured to have a size large enough to support culturing of the embryo, e.g. large enough to prevent waste accumulation to become toxic to the embryo and small enough to prevent excessive dilution of viability signaling factors secreted by the cell. Flow 260 of the culture medium may be intermittent or may be constant at a rate effective to provide necessary nutrients and remove waste by diffusion from the isolation region 458 via connection region 454 of the isolation pen 43. The culture medium may be Continuous Single Culture® Complete (Irvine Scientific), which can be used throughout the entire experiment.

In FIG. 5A, capture beads 574 may be imported into the channel 122, adjacent to the proximal opening 452 of the pen 430 by any appropriate means, including, but not limited to fluid flow 260, gravity, DEP or electrowetting. Embryo secretions 510 exit the pen 430 by diffusion. Typically flow 260 is stopped to prevent movement of the beads 574 and the secretions 510 down the channel 122 and away from the pen 430. Embryo secretions 510 can be captured by the capture beads 574, moved adjacent to the isolation pen 430 or loaded in the isolation pen 430 itself (not shown). The first set of capture beads 574 can be assayed either in place (e.g., in the flow path) or after being exported from the microfluidic device. Alternatively, or in addition, one or more images of the morphology of the embryo 272 can be obtained. Such imaging and/or secretion data can be collected at time 1. Time 1 can be at the single cell zygote stage, as shown in FIG. 5A. Channel walls 414, channel 122, pen 430, isolation region 458, connection region 454, proximal opening 452, and inner surface 208 of FIGS. 5A-C are each as described for FIG. 4.

Subsequently, embryo secretions 512 can be captured by a second set of capture beads 576, located adjacent to the isolation pen 430 or loaded in the isolation pen itself (not shown). Again, the second set of capture beads 576 can be assayed either in place (e.g., in the flow path) or after being exported from the microfluidic device. Alternatively, or in addition, one or more additional images of the morphology of the embryo 274 can be obtained. Such imaging and secretion data can be collected at time 2. Time 2 can be at the two-cell stage, as shown in FIG. 5B. The secretion assay performed with the second set of capture beads 576 can be the same assay as performed with the first set of capture beads 574, or different.

Still later, embryo secretions 514 can be captured by a third set of capture beads 578, moved adjacent to the isolation pen or in the isolation pen itself (not shown). The third set of capture beads 578 can be assayed either in place (e.g., in the flow path) or after being exported from the microfluidic device. Alternatively, or in addition, one or more further images of the morphology of the embryo 276 can be obtained. Such imaging and secretion data can be collected at time 3. Time 3 can be at the four-cell stage, as shown in FIG. 5C. The secretion assay performed with the third set of capture beads 578 can be the same assay as performed with the first (574) or second (576) set of capture beads, or different from one or both of the first and second sets of capture beads. For example, beads 574 may be configured to capture nucleic acid from the single gene responsible for Tay Sachs disease. Analysis off-chip may identify whether the embryo 272 has the targeted mutation. Beads 576 may include a plurality of beads having antibodies to a panel of proteins, including TNF, IL-10, MSP-alpha, SCF, CXCL13, TRAILR3, MIP-1beta, GM-CSF, and one or two housekeeping proteins. The beads may be processed further off chip, or may alternatively, be identified by use of multiplex fluorescently labeled secondary antibodies on chip. If greater proportional fluorescent signal for the panel proteins compared to the housekeeping proteins is found, embryo 274 may be further studied for aneuploidy. One of the sets of beads 574, 574, and 578 may be configured to capture all DNA generally. This set of beads, in one nonlimiting example, could be beads 578, and may be used to determine the ratio of mtDNA to gDNA. This ratio will yield a measure of fitness for the embryo at the 4 cell development time point, and may determine fitness for implantation.

Optionally, embryo secretions and morphology can be monitored periodically until the embryo is at a stage of development suitable for implantation (e.g., a 3-day, 4-day, or 5-day blastula). Based on the secretion and/or morphology data collected, a determination can be made as to whether the embryo is likely to be viable after implantation. Provided that the embryo is likely to be viable (or is predicted to have the highest viability out of a group of embryos, not shown), the embryo 276 (or a subsequent stage of this embryo containing greater numbers of cells) can be implanted into a prospective mother.

### Example 2

### Monitoring, Testing and Conditioning an Ovum Prior to Fertilization

Similarly to Example 1, an ovum 672 can be placed in an isolation region 458 of an isolation pen 430. The microfluidic device can be configured as described for Example 1. Culture medium (G-GAMETE™ (VitroLife)) is flowed in. Flow 260 of culture medium may be intermittent or may be constant at a rate effective to provide necessary nutrients (and remove waste) by diffusion into (or from) the isolation region 458 via connection region 454 of the isolation pen 430. The placement of the ovum 672 into the isolation pen 430 may be performed by fluid flow, gravity, DEP (including OET), electrowetting forces (including OEW), or a combination thereof.

Images may be obtained of the ovum 672 (not shown) and analysis performed on its morphology. Additionally, or alternatively, the sperm to be used in the fertilization may be similarly analyzed, to determine whether the sperm is viable and motile. Based on the results of the analysis, (e.g., one of the ovum or the sperm has defective physiology or function) an activating conditioning is performed during in-vitro fertilization. Sperm may be flowed into channel 122, and are permitted to enter the isolation pen. Activating medium (BTXpress™ Cytoporation™ Media T (Fisher Scientific, part of Thermo Fisher Scientific)) may be flowed in, and replace the initial medium. The activating medium may further contain calcium chloride, in a concentration at about 0.05 mM. An activating ionophore, such as ionomycin or calcimycin, is flowed into the microfluidic channel 122 within about 30 min of the introduction of the sperm, at an effective concentration which may be in the range of about 1 micromolar to about 15 micromolar. After a period of time of exposure to the ionophoric agent, which may be for about 30 min, flow 260 brings in a second medium (e.g., G-FERT™ (VitroLife), to replace the activating medium. The ovum is visualized at this time to determine whether fertilization has occurred. The successfully fertilized ovum can then be monitored and tested as described above in Example 1.

Although specific embodiments and applications of the invention have been described in this specification, these embodiments and applications are exemplary only, and many variations are possible.

1. A process for producing a mammalian embryo in a microfluidic device comprising an isolation pen and a channel configured to contain a fluidic medium, the process comprising:
introducing a mammalian ovum into the isolation pen of the microfluidic device, wherein the isolation pen comprises an isolation region and a connection region, and further wherein a proximal opening of the connection region fluidically connects the isolation region to the channel;
introducing at least one sperm into the microfluidic device;
allowing the at least one sperm to contact the ovum under conditions conducive to fertilization of the ovum; and
incubating the contacted mammalian ovum and the at least one sperm in the microfluidic device for a period of time at least long enough for the mammalian ovum and the at least one sperm to form the embryo.

2. The process of claim 1, wherein introducing the mammalian ovum into the isolation pen comprises using a dielectrophoresis (DEP) force.

3. (canceled)

4. The process of claim 1, wherein introducing the at least one sperm into the isolation pen comprises using a dielectrophoresis (DEP) force.

5.-7. (canceled)

8. The process of claim 1, wherein introducing the ovum into the isolation pen comprises using fluid flow and/or gravity to transport the ovum.

9. The process of claim 1, wherein introducing the at least one sperm into the microfluidic device comprises using fluid flow and/or gravity to transport the at least one sperm.

10. The process of claim 1, further comprising:
ascertaining a status of the mammalian ovum,
wherein the ascertaining is performed prior to introducing the at least one sperm into the microfluidic device.

11. (canceled)

12. The process of claim 1, wherein at least one conditioning treatment is performed on the mammalian ovum prior to introducing the at least one sperm into the microfluidic device.

13. The process of claim 12, wherein the at least one conditioning treatment is an electrical treatment or a chemical treatment.

14.-18. (canceled)

19. The process of claim 1, wherein at least one conditioning treatment is performed on the mammalian ovum after introducing the at least one sperm into the microfluidic device.

20. The process of claim 19, wherein the at least one conditioning treatment is an electrical treatment or a chemical treatment.

21. The process of claim 1, further comprising:
determining that the contacted mammalian ovum and the at least one sperm have formed the embryo.

22. (canceled)

23. The process of claim 1, further comprising performing at least one conditioning treatment on the embryo.

24.-25. (canceled)

26. The process of claim 21, wherein determining that the embryo has formed comprises detecting secretions within or coming from the isolation pen in which the mammalian ovum was introduced.

27. The process of claim 26, wherein the detection of secretions comprises detecting protein or nucleic acid.

28. (canceled)

29. The process of claim 1, wherein the isolation pen contains a single mammalian ovum.

30. The process of claim 1, wherein the microfluidic device contains a plurality of isolation pens, and further wherein a single mammalian ovum is introduced into each of two or more isolation pens of the plurality.

31.-36. (canceled)

37. The process of claim 1, further comprising:
exporting the embryo out of the microfluidic device.

38.-39. (canceled)

40. A process for monitoring a status of at least one biological micro-object in a microfluidic device comprising an isolation pen and a channel configured to contain a fluidic medium, wherein the biological micro-object is selected from an embryo, sperm or ovum, the process comprising:
introducing the biological micro-object into the isolation pen of the microfluidic device, wherein the isolation pen comprises an isolation region and a connection region, and further wherein a proximal opening of the connection region fluidically connects the isolation region to the channel;
providing a medium to the biological micro-object configured to provide nutrients necessary for viability, wherein the provided medium comprises components necessary to activate the biological micro-object for a subsequent biological transformation;
analyzing a secretion produced by the biological micro-object; and
determining the status of the biological micro-object.

41.-42. (canceled)

43. The process of claim 40, wherein the step of analyzing the secretion comprises capturing the secretion with a capture bead.

44.-45. (canceled)

46. The process of claim 40, wherein analyzing the secretion comprises detecting proteins, nucleic acids, fragments of any of the foregoing, or any combination thereof.

47.-48. (canceled)

49. The process of claim 40, further comprising:
imaging the biological micro-object.

50. (canceled)

51. The process of claim 40, wherein the biological micro-object is an embryo, and wherein the embryo is exported after determining that the embryo is viable.

52. (canceled)

53. A process for monitoring a status of at least one biological micro-object in a microfluidic device comprising an isolation pen and a channel configured to contain a fluidic medium, wherein the biological micro-object is selected from a mammalian embryo, sperm or ovum, the process comprising:
introducing the biological micro-object into the isolation pen of the microfluidic device, wherein the isolation pen comprises an isolation region and a connection region, and further wherein a proximal opening of the connection region fluidically connects the isolation region to the channel;
providing a medium to the biological micro-object; configured to provide nutrients necessary for viability;
imaging the biological micro-object; and
determining the status of the biological micro-object.

54.-56. (canceled)

57. The process of claim 53, wherein the step of determining the status comprises determining a size, a shape or both of a mammalian ovum.

58. The process of claim 57, further comprising determining whether to perform a conditioning treatment on the mammalian ovum based on the determined size and/or determined shape of the mammalian ovum.

59. (canceled)

60. The process of claim 53, wherein the step of determining the status comprises determining at least one of size, shape, motility and chemotactic responses of a mammalian sperm.

61.-62. (canceled)

63. The process of claim 53, wherein the step of determining the status comprises determining at least one of a size, a shape, and a timing of cell divisions of an embryo.

64.-83. (canceled)
